(19) United States
(12) Patent Application Publication
(10) Pub. No.: US 2017/0283789 A1
(43) Pub. Date: Oct. 5, 2017

(54) METHODS FOR COLLECTING CERVICAL-VAGINAL FLUIDS AND ISOLATING EXOSOME AND MICROVESICLES FOR MOLECULAR ANALYSIS

(71) Applicants: HITACHI CHEMICAL CO., LTD., Tokyo (JP); HITACHI CHEMICAL CO. AMERICA, LTD., San Jose, CA (US)

(72) Inventors: Cindy M. Yamamoto, Irvine, CA (US); Vivian Tovar, Santa Ana, CA (US); Taku Murakami, Irvine, CA (US); Mieko Ogura, Newport Coast, CA (US); Melanie Oakes, San Juan Capistrano, CA (US); Masato Mitsuhashi, Irvine, CA (US)

(21) Appl. No.: 15/507,218

(22) PCT Filed: Aug. 27, 2015
(86) PCT No.: PCT/US15/47110

§ 371 Date: Feb. 27, 2017

Related U.S. Application Data
(60) Provisional application No. 62/044,029, filed on Aug. 29, 2014.

(51) Int. Cl. C12N 15/10; C12Q 1/68

(52) U.S. Cl. CPC C12N 15/1003; C12Q 1/6886; C12Q 1/6806; C12Q 1/6872; C12Q 1/6874; C12N 15/1072; C12Q 2600/156; C12Q 1/68; C12Q 2600/158

(57) ABSTRACT

The present disclosure relates to methods of collecting cervical-vaginal fluid exosomes and microvesicles and isolating corresponding mRNA. In particular, certain embodiments relate to the method of collecting the cervical-vaginal fluids with a tampon and releasing the cells, exosomes and microvesicles using excess buffer and a syringe or syringe-like device. The resulting expunged fluid can be applied to a filter device that is capable of capturing exosomes and microvesicles. Nucleic acids such as mRNA can be isolated from the cervical-vaginal fluid exosome and microvesicles using an oligo(dT)-coated plate designed to accommodate the filter device and then used for further molecular analysis. Quantification of the collected nucleic acids may then be used in the diagnosis and/or treatment of gynecological diseases or conditions.

### RELATED CASES

Any and all applications for which a foreign or domestic priority claim is identified in the Application Data Sheet as filed with the present application are hereby incorporated by reference.

### BACKGROUND

### Field

Embodiments of the invention relate generally to methods of identifying and/or treating patients with gynecological-related diseases and conditions. In particular, several embodiments relate to quantification of RNA isolated from exosomes, vesicles, or other biological components of interest that have been collected from a patient's cervical-vaginal fluid.

### Description of Related Art

The female reproductive system is in constant flux beginning from puberty to menopause based on the normal reproductive cycles, pregnancy, aging and sometimes disease or injury. Female reproductive organs in the abdominal and pelvic area include the uterus, ovaries, fallopian tubes, vagina and vulva. Various diseases and conditions that can present in or from these female reproductive organs include cancer, endometriosis, fibroids, Polycystic Ovarian Syndrome and vulvodynia. Although there are currently two effective screening tests (Papanicolaou and human papilloma virus test) for cervical cancer, other gynecological cancers such as ovarian, vaginal and vulvar cancers do not have a simple and reliable way of screening. Conditions such as endometriosis, fibroids, PCOS and vulvodynia do not have known causes and diagnosis is not always simple, typically involving pelvic exams, ultrasound and/or laparoscopy.

There is an immediate need to develop early screening methods for certain gynecological cancers. Ovarian cancer causes more deaths than any other female reproductive cancer, but if it is found at a localized stage, about 94% of patients live longer than 5 years after diagnosis. As a result, much research has been done to identify screening tests, but none have been very successful. A substance released from epithelial ovarian cancer cells called carbohydrate antigen (CA-125) was found to be a possible marker for screening, but was recently shown to have no effect on decreasing the number of deaths from this disease. Transvaginal ultrasound is an imaging technique that was found to be a promising routine screening test but is not able to distinguish between a benign and cancer mass. The prostate, lung, colorectal, ovarian (PLCO) trial evaluating the combined use of serum CA-125 and transvaginal ultrasound found that among women in the U.S., simultaneous screening did not reduce mortality rates from ovarian cancer compared to standard of care.

### SUMMARY

Exosomes and microvesicles can be isolated from various biological fluids such as urine, blood and saliva. The RNA enclosed within these biological components is protected from degradation by nucleases and could be used as potential non-invasive sources of biomarkers. In several embodiments, the present disclosure relates to methods of collecting cervical-vaginal fluids and isolating exosomes and microvesicles in order to identify biomarkers with improved sensitivity for detecting gynecological diseases and other conditions. In particular, in some embodiments methods of collecting cervical-vaginal fluids and identifying biomarkers for gynecological diseases and conditions are provided. In some embodiments, the method comprises: obtaining cervical-vaginal fluids by usage of female sanitary products (e.g., a tampon, pantiliners, sanitary pads, etc.), isolating one or more of membrane particles, cells, exosomes, exosome-like vesicles, and microvesicles from the sanitary product containing cervical-vaginal fluids, and detecting expression of a biomarker. In several embodiments, the detected biomarker is then used to develop an appropriate treatment regimen. In several embodiments, however, the treatment may be taking no further action (e.g., not instituting a treatment). In some embodiments, expression of a biomarker is detected by a method comprising liberating RNA from the isolated membrane particles, cells, exosomes, exosome-like vesicles, and/or microvesicles, contacting the liberated RNA with a reverse transcriptase to generate complementary DNA (cDNA), and contacting the cDNA with sense and antisense primers that are specific for the biomarker of the gynecological disease or condition and a DNA polymerase in order to generate amplified DNA. In several embodiments the methods are computerized (e.g., one or more of the RNA isolation, cDNA generation, or amplification are controlled, in whole or in part, by a computer). In several embodiments, the detection of the biomarker is real time.

In additional embodiments, a method of collecting cervical-vaginal fluids and identifying biomarkers for gynecological diseases and conditions is provided. In some embodiments, the method comprises obtaining cervical-vaginal fluids by tampon usage (or another type of female sanitary product), isolating one or more of membrane particles, cells, exosomes, exosome-like vesicles, and/or microvesicles from the tampon containing cervical-vaginal fluids, and identifying mRNA expression profile or mutations. In some embodiments, the method of identifying mRNA expression profile or mutations comprises one or more of liberating RNA from the isolated membrane particles, cells, exosomes, exosome-like vesicles, and/or microvesicles, preparing double-stranded cDNA library for RNA sequencing, and performing RNA sequencing.

In some embodiments, a method of collecting vaginal fluids and analyzing biomarkers to determine whether a subject is suffering from a gynecological disease or condition is provided. In some embodiments, the method comprises obtaining a tampon (or other female sanitary product) comprising cervical-vaginal fluids that has been used by a subject, isolating one or more of membrane particles, cells, exosomes, exosome-like vesicles, and microvesicles from the tampon, detecting expression of a biomarker associated with a gynecological disease or condition, comparing the expression of the biomarker to expression of the biomarker in a control sample, and determining that a subject is suffering from a gynecological disease or condition when expression of the biomarker is greater when compared to the expression in a control sample. In some embodiments, detecting the expression of a biomarker comprises the steps of: liberating RNA from the isolated membrane particles, cells, exosomes, exosome-like vesicles, and/or microvesicles, contacting the liberated RNA with a reverse transcriptase to generate complementary DNA (cDNA), and contacting the cDNA with sense and antisense primers that are specific for the biomarker and a DNA polymerase in order to generate amplified DNA. In several embodiments the methods are computerized (e.g., one or more of the RNA isolation, cDNA generation, or amplification are controlled, in whole or in part, by a computer). In several embodiments, the detection of the biomarker is real time.

In other embodiments, a method of collecting cervical-vaginal fluids from a subject to determine whether a subject has a gynecological disease or condition, comprises obtaining a tampon (or other female sanitary product) comprising cervical-vaginal fluids that has been used by a subject, isolating one or more of membrane particles, cells, exosomes, exosome-like vesicles, and microvesicles from the tampon, identifying an mRNA expression profile, and determining whether a subject has a gynecological disease or condition based on comparing the mRNA expression profile of the subject to a control mRNA expression profile. In some embodiments, the mRNA expression profile is identified by liberating RNA from the isolated membrane particles, cells, exosomes, exosome-like vesicles, and microvesicles, preparing a double-stranded cDNA library for RNA sequencing from the RNA, performing sequencing of the cDNA, identifying the mRNA expression profile based on the results of the cDNA sequencing. In several embodiments the methods are computerized (e.g., one or more of the identification of the mRNA expression profile, the comparison of the mRNA expression profile to a control profile, RNA, isolation, cDNA generation, amplification, or DNA sequencing are controlled, in whole or in part, by a computer). In several embodiments, the detection of the biomarker is real time. In several embodiments, the DNA sequencing is performed using Next Generation Sequencing methods.

In some embodiments, a method of treating a subject suffering from a gynecological disease or condition is disclosed. In some embodiments, the method comprises: ordering that a tampon (or other female sanitary product) with cervical-vaginal fluid that has been used by a subject, ordering a test of the cervical-vaginal fluid of the tampon, obtaining the results of the test, and treating the subject when the test results indicate that the subject is suffering from a gynecological disease or condition. In some embodiments, the test comprises the steps of isolating one or more of membrane particles, cells, exosomes, exosome-like vesicles, and/or microvesicles from the cervical-vaginal fluid of the tampon, liberating RNA from the isolated membrane particles, cells, exosomes, exosome-like vesicles, and microvesicles, contacting the liberated RNA with a reverse transcriptase to generate complementary DNA (cDNA), contacting the cDNA with sense and antisense primers that are specific for one or more markers of gynecological disease or condition and a DNA polymerase in order to generate amplified DNA, and detecting the amount of expression of the markers of gynecological disease or condition. As above, certain aspects of the methods are optionally computerized. Also, in several embodiments, the amount of expression may result in a determination that no treatment is to be undertaken at that time. Thus, in several embodiments, the methods disclosed herein also reduce unnecessary medical expenses and reduce the likelihood of adverse effects from a treatment that is not needed at that time.

In other embodiments, a method of treating a subject suffering from a gynecological disease or condition comprises the steps of obtaining a tampon (or other female sanitary product) comprising cervical-vaginal fluids that has been used by a subject, isolating one or more of membrane particles, cells, exosomes, exosome-like vesicles, and microvesicles from the tampon, detecting expression of a biomarker associated with a gynecological disease or condition, comparing the expression of the biomarker to expression of the biomarker in a control sample, treating the subject for a gynecological disease or condition when expression of the biomarker is different when compared to the expression in the control sample. In some embodiments, detecting expression of the biomarker comprises the method of liberating RNA from the isolated membrane particles, cells, exosomes, exosome-like vesicles, and/or microvesicles, contacting the liberated RNA with a reverse transcriptase to generate complementary DNA (cDNA), and contacting the cDNA with sense and antisense primers that are specific for the biomarker and a DNA polymerase in order to generate amplified DNA.

In still additional embodiments, a method of treating a subject suffering from a gynecological disease or condition comprises the steps of obtaining a tampon (or other female sanitary product) comprising cervical-vaginal fluids that has been used by a subject, isolating one or more of membrane particles, cells, exosomes, exosome-like vesicles, and microvesicles from the tampon, identifying an mRNA expression profile, and treating the subject for a gynecological disease or condition when the mRNA expression profile of the subject is different when compared to a control mRNA expression profile. In some embodiments, an mRNA expression profile is identified by a method comprising liberating RNA from the isolated membrane particles, cells, exosomes, exosome-like vesicles, and microvesicles, preparing a double-stranded cDNA library for RNA sequencing from the RNA, performing sequencing of the cDNA, and identifying the mRNA expression profile based on the results of the cDNA sequencing.

In some embodiments, a method of directing treatment of a subject suffering from a gynecological condition or disease is disclosed. In some embodiments, the method comprises: receiving cervical-vaginal fluid collected from a tampon that has been used by a subject, detecting expression of at least one marker of gynecological disease or condition, identifying the subject as suffering from a gynecological disease or condition when the expression of the marker is different as compared to a control mRNA expression profile, informing a physician that it would be appropriate to treat the subject if the expression indicates that the subject is suffering from a gynecological disease or condition (or not treat the subject if no disease or condition is detected based on the expression of the markers). In some embodiments, detecting expression of at least one marker comprises the steps of isolating one or more of membrane particles, cells, exosomes, exosome-like vesicles, and microvesicles from the cervical-vaginal fluid of the tampon, liberating RNA from the isolated membrane particles, cells, exosomes, exosome-like vesicles, and microvesicles, contacting the liberated RNA with a reverse transcriptase to generate complementary DNA (cDNA), and contacting the cDNA with sense and antisense primers that are specific for the marker of gynecological disease in order to generate amplified DNA. In several embodiments, at least a portion of the methods are computerized (e.g., one or more of the RNA isolation, cDNA generation, or amplification are controlled, in whole or in part, by a computer). In several embodiments, the detection of the biomarker is real time. Additionally, in several embodiments, the informing is performed by computer or other form of network communication. In several such embodiments, the computers (or tablets, smartphones, etc.) involved in transmitting or receiving of the expression information comprise a graphical user interface that provides the physician with therapeutic options for treating the subject, when appropriate and allows the physician to filter or otherwise refine the information provided based on therapeutic preferences derived from characteristics specific to the subject.

In some embodiments, a method of collecting vaginal fluids and analyzing biomarkers to determine whether a subject is suffering from a gynecological disease or condition is disclosed. In some embodiments, the method comprises obtaining a tampon (or other female sanitary product) comprising cervical-vaginal fluids that has been used by a subject, isolating one or more of membrane particles, cells, exosomes, exosome-like vesicles, and microvesicles from the tampon, detecting expression of a biomarker associated with a gynecological disease or condition, and using a computer configured to receive data regarding the expression of the biomarker and programmed to determine whether the expression of the biomarker indicates that a subject is suffering from a gynecological disease or condition. In some embodiments, detecting expression of the biomarker comprises the steps of: liberating RNA from the isolated membrane particles, cells, exosomes, exosome-like vesicles, and/or microvesicles, contacting the liberated RNA with a reverse transcriptase to generate complementary DNA (cDNA), and contacting the cDNA with sense and antisense primers that are specific for the biomarker and a DNA polymerase in order to generate amplified DNA.

In some embodiments, the cervical-vaginal fluids are collected from a feminine hygiene product. In some embodiments, the cervical-vaginal fluids are collected from a tampon. In other embodiments, the cervical-vaginal fluids are collected from a pantiliner or pad. In other embodiments, the cervical-vaginal fluids are collected during a visit to a physician, for example, during a pap-smear. In still additional embodiments, cervical-vaginal fluids that would otherwise be considered bio-waste material are used as a source material for determining whether a subject is afflicted with a gynecological disease. For example, effluent from a douche procedure (either home-use or performed in a medical facility) can be a source of nucleic acids for measuring marker expression. In some embodiments, fluids obtained from intravaginal procedures (e.g., dilation and curettage, ultrasounds, etc.) can be a source of nucleic acids for measuring marker expression.

In some embodiments, the cervical-vaginal fluids are collected from a tampon comprised of cotton and rayon fibers. In some embodiments, only cotton or only rayon fibers are present in the tampon. In some embodiments, the tampon can absorb up to 6 grams of fluid. In other embodiments, the tampon can absorb from about 2 g to about 3 g, about 3 g to about 4 g, about 4 g to about 5 g, about 5 g to about 6 g, about 6 g to about 7 g, about 7 g to about 8 g, or any volume in between these ranges.

In some embodiments, the cervical-vaginal fluids are collected from a feminine hygiene product after the product has been used by a subject for up to eight hours during a non-menstrual time-period. In some embodiments, the cervical-vaginal fluids are collected from a feminine hygiene product after the product has been used by a subject during a menstrual period. In other embodiments, the cervical-vaginal fluids are collected after the feminue hygiene product is used for about 1 hour to about 2 hours, about 2 hours to about 3 hours, about 3 hours to about 4 hours, about 4 hours to about 5 hours, about 5 hours to about 6 hours, about 6 hours to about 7 hours, about 7 hours to about 8 hours, about 8 hours to about 9 hours, about 9 hours to about 10 hours or any range in between.

In some embodiments, membrane particles, cells, exosomes, exosome-like vesicles, microvesicles, and/or any other nucleic acid-containing cell or fragment thereof are isolated from the female hygiene product. In some embodiments, any of these materials are isolated by adding an excess of a buffer to the tampon. In some embodiments, between 10 to 30 mL of the excess buffer is added to the tampon. In some embodiments, the amount of excess buffer added is between about 10 mL to about 15 mL, about 15 mL to about 20 mL, about 20 mL to about 25 mL, about 25 mL to about 30 mL, or any range in between. In some embodiments, the excess buffer is composed of pH greater than 4 and less than 10. In some embodiments, the excess buffer is a pH of between about 4 and about 5, about 5 and about 6, about 6 and about 7, about 7 and about 8, about 8 and about 9, about 9 and about 10, or any pH between these ranges. In some embodiments, the excess buffer does not comprise a detergent. In some embodiments, the excess buffer does comprise a detergent.

In some embodiments, the membrane particles, cells, exosomes, exosome-like vesicles, and/or microvesicles are isolated from the tampon using one or more types of physical force. In some embodiments, a syringe or syringe-like device is used to isolate the material (e.g., via suction or, alternatively, via positive pressure). In other embodiments, centrifugation, shaking, air pressure, or liquid pressure are used. Combinations may also be used, in several embodiments.

In some embodiments, after a biological sample is collected from the female hygiene product, membrane particles, cells, exosomes, exosome-like vesicles, microvesicles and/or other biological components of interest are isolated from the tampon by filtering the sample. In some embodiments, filtering the collected sample will trap one or more of membrane particles, exosomes, exosome-like vesicles, and microvesicles on a filter. In some embodiments, the filter comprises material to capture components that are about 1.6 microns or greater in diameter. In several embodiments, a plurality of filters are used to capture vesicles within a particularly preferred range of sizes (e.g., diameters). For example, in several embodiments, filters are used to capture vesicles having a diameter of from about 0.2 microns to about 1.6 microns in diameter, including about 0.2 microns to about 0.4 microns, about 0.4 microns to about 0.6 microns, about 0.6 microns to about 0.8 microns, about 0.8 microns to about 1.0 microns, about 1.0 microns to about 1.2 microns, about 1.2 to about 1.4 microns, about 1.4 microns to about 1.6 microns (and any size in between those listed). In other embodiments, the vesicle-capture material captures exosomes ranging in size from about 0.5 microns to about 1.0 microns.

In some embodiments, the filter (or filters) comprises glass-like material, non-glass-like material, or a combination thereof. In some embodiments, wherein the vesicle-capture material comprises glass-like materials, the vesicle-capture material has a structure that is disordered or “amorphous” at the atomic scale, like plastic or glass. Glass-like materials include, but are not limited to glass beads or fibers, silica beads (or other configuration), nitrocellulose, nylon, polyvinylidene fluoride (PVDF) or other similar polymers, metal or nano-metal fibers, polystyrene, ethylene vinyl acetate or other co-polymers, natural fibers (e.g., silk), alginate fiber, or combinations thereof. In certain embodiments, the vesicle-capture material optionally comprises a plurality of layers of vesicle-capture material. In other embodiments, the vesicle-capture material further comprises nitrocellulose.

In some embodiments, a filter device is used to isolate biological components of interest. In some embodiments, the device comprises: a first body having an inlet, an outlet, and an interior volume between the inlet and the outlet; a second body having an inlet, an outlet, an interior volume between the inlet and the outlet, a filter material positioned within the interior volume of the second body and in fluid communication with the first body; and a receiving vessel having an inlet, a closed end opposite the inlet and interior cavity. In some embodiments, the first body and the second body are reversibly connected by an interaction of the inlet of the second body with the outlet of the first body. In some embodiments, the interior cavity of the receiving vessel is dimensioned to reversibly enclose both the first and the second body and to receive the collected sample after it is passed from the interior volume of the first body, through the filter material, through the interior cavity of the second body and out of the outlet of the second body. In some embodiments, the isolating step comprises placing at least a portion of the collected sample in such a device, and applying a force to the device to cause the collected sample to pass through the device to the receiving vessel and capture the biological component of interest. In some embodiments, applying the force comprises centrifugation of the device. In other embodiments, applying the force comprises application of positive pressure to the device. In other embodiments, applying the force comprises application of vacuum pressure to the device. Examples of such filter devices are disclosed in PCT Publication WO 2014/182330 and PCT Publication WO 2015/050891, hereby incorporated by reference herein.

In some embodiments, the collected sample is passed through multiple filters to isolate the biological component of interest. In other embodiments, isolating biological components comprises diluting the collected sample. In other embodiments, centrifugation may be used to isolate the biological components of interest. In some embodiments, multiple isolation techniques may be employed (e.g., combinations of filtration selection and/or density centrifugation). In some embodiments, the collected sample is separated into one or more samples after the isolating step.

In some embodiments, RNA is liberated from the biological component of interest for measurement. In some embodiments, liberating the RNA from the biological component of interest comprises lysing the membrane particles, exosomes, exosome-like vesicles, and/or microvesicles with a lysis buffer. In other embodiments, centrifugation may be employed. In some embodiments, the liberating is performed while the membrane particles, exosomes, exosome-like vesicles, microvesicles and/or other components of interest are immobilized on a filter. In some embodiments, the membrane particles, exosomes, exosome-like vesicles, microvesicles and/or other components of interest are isolated or otherwise separated from other components of the collected sample (and/or from one another—e.g., vesicles separated from exosomes).

In some embodiments, expression is detected, analyzed, or identified for one or more biomarkers selected from the group consisting of IL8, FTL, B2M, S100A8, SAT1, IFITM2, S100A9, SPRR3, SOD2, FTH1, IFI30, H3F3B, BCL2A1, LITAF, FCER1G, ACTB, S100A1, GOS2, SRGN, LCE3D, GLUL, PI3, IL1B, IFITM3, IL1RN, CCL4, CYSTM1, SDCBP, PLEK, EIF1, CNFN, ANXA1, MYL6, GAPDH, Cl5orf48, KRT13, RGS2, SPRR1B, NOP10, GABARAP, TYROBP, PLAUR, SPRR2D, FPR1, SPRR2A, TMSB4X, TIMP1, FAM25A, CRCT1, GABARAPL2, RHOA, SLPI, ACTG1, ALOX5AP, LAPTM5, IFITM1, CXCL1, CSTB, CARD16, S100A12, NINJ1, AIF1, S100A7, AQP9, ARHGDIB, CCL3, IGSF6, NAMPT, CASP4, MNDA, LCP1, SAMSN1, ALDOA, CLIC1, SH3BGRL3, PNRC1, SPRR1A, TPI1, SERPINA1, TALDO1, LST1, LINC01272, GMFG, CRNN, CD53, TAGLN2, LY96, RAC2, IVNS1ABP, ISG20, PLSCR1, TPT1, MYL12A, LDHA, LCN2, S100A6, MXD1, SPINK7, RPLP1, UBE2B, CXCL8, DUSP1, RPL23, RPS11, PROK2, RPL27, CXCL2, ZFP36L1, BASP1, CSTA, FOX, PCBP1, RPL38, BRI3, SDCBP, CCL20, RPS12, RPL37A, CEBPB, SPRR2E, NFKBIA, RPL30, RPL24, CYSTM1, RGS2, RPS25, CXCR4, C4orf3, PABPC1, S100P, RPL26, GCA, MARCKS, RPS27A, SELK, ITM2B, MAL, HSPA1A, RPS29, PPP1CB, RPS20, IVNS1ABP, ZFP36, and TXN. 28. In some embodiments, the biomarker is selected from the group consisting of IL-8, FTL, B2M, S100A8, S100A9, SAT1, IFITM2, SPRR3, SOD2, FTH1, CXCL8, GOS2, SRGN, IL-1B, and CXCL1.

In some embodiments, the RNA liberated from the biological components of interest comprises poly(A)+RNA.

According to various embodiments, various methods to quantify RNA are used, including Northern blot analysis, RNAse protection assay, PCR, RT-PCR, real-time RT-PCR, other quantitative PCR techniques, RNA sequencing, nucleic acid sequence-based amplification, branched-DNA amplification, mass spectrometry, CHIP-sequencing, DNA or RNA microarray analysis and/or other hybridization microarrays. In some of these embodiments or alternative embodiments, after amplified DNA is generated, it is exposed to a probe complementary to a portion of a biomarker of interest.

In some embodiments, a computerized method is used to complete one or more of the steps. In some embodiments, the computerized method comprises exposing a reaction mixture comprising isolated RNA and/or prepared cDNA, a polymerase and gene-specific primers to a thermal cycle. In some embodiments, the thermal cycle is generated by a computer configured to control the temperature time, and cycle number to which the reaction mixture is exposed. In other embodiments, the computer controls only the time or only the temperature for the reaction mixture and an individual controls on or more additional variables. In some embodiments, a computer is used that is configured to receive data from the detecting step and to implement a program that detects the number of thermal cycles required for the biomarker to reach a pre-defined amplification threshold in order to identify whether a subject is suffering from a gynecological disease or condition. In still additional embodiments, the entire testing and detection process is automated.

For example, in some embodiments, RNA is isolated by a fully automated method, e.g., methods controlled by a computer processor and associated automated machinery. In one embodiment a biological sample, such as biological material on a tampon, is collected and loaded into a receiving vessel that is placed into a sample processing unit. A user enters information into a data input receiver, such information related to sample identity, the sample quantity, and/or specific patient characteristics. In several embodiments, the user employs a graphical user interface to enter the data. In other embodiments, the data input is automated (e.g., input by bar code, QR code, or other graphical identifier). The user can then implement an RNA isolation protocol, for which the computer is configured to access an algorithm and perform associated functions to process the sample in order to isolate biological components, such as vesicles, and subsequently processed the vesicles to liberate RNA. In further embodiments, the computer implemented program can quantify the amount of RNA isolated and/or evaluate and purity. In such embodiments, should the quantity and/or purity surpass a minimum threshold, the RNA can be further processed, in an automated fashion, to generate complementary DNA (cDNA). cDNna can then be generated using established methods, such as for example, binding of a poly-A RNA tail to an oligo dT molecule and subsequent extension using an RNA polymerase. In other embodiments, if the quantity and/or purity fail to surpass a minimum threshold, the computer implemented program can prompt a user to provide additional biological sample(s).

Depending on the embodiment, the cDNA can be divided into individual subsamples, some being stored for later analysis and some being analyzed immediately. Analysis, in some embodiments comprises mixing a known quantity of the cDNA with a salt-based buffer, a DNA polymerase, and at least one gene specific primer to generate a reaction mixture. The cDNA can then be amplified using a predetermined thermal cycle program that the computer system is configured to implement. This thermal cycle, could optionally be controlled manually as well. After amplification (e.g., real-time PCR,), the computer system can assess the number of cycles required for a gene of interest (e.g. a marker of gynecological disease or condition) to surpass a particular threshold of expression. A data analysis processor can then use this assessment to calculate the amount of the gene of interest present in the original sample, and by comparison either to a different patient sample, a known control, or a combination thereof, expression level of the gene of interest can be calculated. A data output processor can provide this information, either electronically in another acceptable format, to a test facility and/or directly to a medical care provider. Based on this determination, the medical care provider can then determine if and how to treat a particular patient based on determining the presence of a gynecological disease or condition. In several embodiments, the expression data is generated in real time, and optionally conveyed to the medical care provider (or other recipient) in real time.

In several embodiments, a fully or partially automated method enables faster sample processing and analysis than manual testing methods. In certain embodiments, machines or testing devices may be portable and/or mobile such that a physician or laboratory technician may complete testing outside of a normal hospital or laboratory setting. In some embodiments, a portable assay device may be compatible with a portable device comprising a computer such as a cell phone or lap top that can be used to input the assay parameters to the assay device and/or receive the raw results of a completed test from the assay device for further processing. In some embodiments, a patient or other user may be able to use an assay device via a computer interface without the assistance of a laboratory technician or doctor. In these cases, the patient would have the option of performing the test “at-home.” In certain of these embodiments, a computer with specialized software or programming may guide a patient to properly place a sample in the assay device and input data and information relating to the sample in the computer before ordering the tests to run. After all the tests have been completed, the computer software may automatically calculate the test results based on the raw data received from the assay device. The computer may calculate additional data by processing the results and, in some embodiments, by comparing the results to control information from a stored library of data or other sources via the internet or other means that supply the computer with additional information. The computer may then display an output to the patient (and/or the medical care provider, and/or a test facility) based on those results.

In some embodiments, a medical professional may be in need of genetic testing in order to diagnose, monitor and/or treat a patient. Thus, in several embodiments, a medical professional may order a test and use the results in making a diagnosis or treatment plan for a patient. For example, in some embodiments a medical professional may collect a sample from a patient or have the patient otherwise provide a sample (or samples) for testing. The medical professional may then send the sample to a laboratory or other third party capable of processing and testing the sample. Alternatively, the medical professional may perform some or all of the processing and testing of the sample himself/herself (e.g., in house). Testing may provide quantitative and/or qualitative information about the sample, including data related to the presence of a gynecological disease. Once this information is collected, in some embodiments the information may be compared to control information (e.g., to a baseline or normal population) to determine whether the test results demonstrate a difference between the patient's sample and the control. After the information is compared and analyzed, it is returned to the medical professional for additional analysis. Alternatively, the raw data collected from the tests may be returned to the medical professional so that the medical professional or other hospital staff can perform any applicable comparisons and analyses. Based on the results of the tests and the medical professional's analysis, the medical professional may decide how to treat or diagnose the patient (or optionally refrain from treating).

In some embodiments, expression of a biomarker is compared to expression of the biomarker in a control sample. In some embodiments, the control sample is based on the expression of the biomarker in a healthy individual, or an individual who is not suffering from a gynecological disease or condition. In other embodiments, the control sample is based on an average or control RNA expression profile generated based on the average biomarker expression of multiple healthy individuals. In other embodiments, the control sample is based on the expression of the biomarker in an individual who is suffering from a gynecological disease or condition. In other embodiments, the control sample is generated by a computer that has received data for subjects whose biomarker expression levels have been analyzed. In some embodiments, multiple samples are taken from the same individual at different times over the course of days, weeks, months, or years. In these embodiments, the earlier data collected may be used to generate a control sample to compare to the later data. In addition, these multiple samples can be used to track whether (and how) mRNA expression changes in a patient over time.

In some embodiments, an mRNA expression profile is generated for one or more mRNA associated with a gynecological disease or condition or any other biomarkers. In some embodiments, the mRNA expression profile may be generated to include a comparison of the expression of a biomarker in an individual to the expression of the biomarker in a control sample, where the control sample is generated by any of the methods described above or through alternative means that similarly provide a data reference point. In some embodiments, an mRNA expression profile may be based on mRNA data collected from the individual patient alone, where expression data was collected on either one or multiple occasions.

In some embodiments, greater expression of a biomarker indicates a subject is suffering from a gynecological disease or condition. In other embodiments, reduced expression of a biomarker indicates a subject is suffering from a gynecological disease or condition. Depending on the marker, and the embodiment, increases or decreases in expression may be statistically significant (e.g., p-values less than 0.05 by art-accepted statistical analysis methods). In some embodiments, expression is compared against a control value or expression profile to determine whether a subject is suffering from a gynecological disease or condition compared to the control. In some embodiments, expression indicating gynecological disease or condition or lack thereof is corroborated with a histological evaluation of a biopsy of a cell or tissue population of interest.

In some embodiments, the results of a test indicate that a subject is suffering from a gynecological disease or condition when an expression of a biomarker is different than a control mRNA expression profile. In some embodiments, the difference in the expression profile of the subject as compared to a control mRNA expression profile is an increase in the expression of a biomarker associated with a gynecological disease or condition. In other embodiments, the difference in the expression profile of the subject as compared to a control mRNA expression profile is a decrease in the expression of a biomarker associated with a gynecological disease or condition. In some embodiments, the results of a test indicate that a subject is suffering from a gynecological disease or condition when an expression of a biomarker is the same as a control mRNA expression profile.

In some embodiments, the gynecological disease or condition is cancer or a pre-cancerous disease. In some embodiments, the gynecological disease or condition is cancer or a pre-cancerous disease of the ovaries, fallopian tubes, uterus, cervix, vagina, vulva, or other reproductive organs. In some embodiments, the gynecological disease or condition is incontinence of urine, amenorrhoea (or absent menstrual periods), dysmenorrhea (painful menstrual periods), infertility, menorrhagia (heavy menstrual periods, or prolapse of pelvic organs. In some embodiments, the gynecological disease or condition is a fungal, bacterial, viral, or protozoal infection. In some embodiments, the infection affects the vagina, cervix, or uterus.

In some embodiments, the gynecological disease or condition is treated with oral or topical medication. Medications are not limited to a compound that is generally considered of medicinal purpose (e.g., a prescribed or over the counter drug) but may also include any dietary or nutrition supplement(s). Therefore, for example, a vitamin, a mineral, an herb or other botanical, an amino acid, a dietary substance for use by a subject to supplement the diet by increasing the total dietary intake (e.g., enzymes or tissues from organs or glands), or a concentrate, metabolite, constituent or extract can also be applicable to the methods disclosed herein. In other embodiments, the gynecological disease or condition is treated with surgery or further inspection of the subject, such as with ultrasound. In some embodiments, the subject is treated using a hysterectomy, oophorectomy, or tubal ligation.

The methods summarized above and set forth in further detail below describe certain actions taken by a practitioner; however, it should be understood that they can also include the instruction of those actions by another party. Thus, actions such as “treating a subject for a gynecological disease or condition” include “instructing the administration of treatment of a subject for a gynecological disease or condition.”

### BRIEF DESCRIPTION OF THE FIGURES

FIGS. 1A and 1B show expression data related to various methods of collecting exosomes and microvesicles.

FIGS. 2A-2D show various magnifications of scanning electron microscopy images of cervical-vaginal fluid exosomes and microvesicles.

FIGS. 3, 3-1 and 3-2 are a table listing genes that are highly expressed in cervical-vaginal fluid exosomes and microvesicles based on Clontech Single Cell and Low Input library preparation.

FIG. 4 is a schematic diagram of the RNA amplification process using a capture plate.

FIGS. 5, 5-1 and 5-2 are a table listing genes that are highly expressed in cervical-vaginal fluid exosomes and microvesicles based on T7 amplified RNA and TruSeq library preparation.

### DETAILED DESCRIPTION

Early screening methods for gynecological diseases and conditions should be non-invasive, simple, low cost and provide results with high sensitivity and specificity. As described above, current biomarkers for ovarian cancer such as CA-125 and transvaginal ultrasound have not been successful in reducing mortality rates. Novel biomarkers, in particular for ovarian cancer, have been difficult to identify. Human epididymis protein 4 (HE4) is a new marker associated with ovarian cancer but is not disease-specific. Similar to CA-125, the HE4 immunoassay is not used for cancer screening, but is an additional tool that can be used to monitor therapeutic response and/or detect ovarian cancer recurrence. Other novel biomarkers are currently being studied and include miRNAs and DNA methylation patterns in groups of genes regulated during ovarian cancer progression.

Several embodiments of the methods disclosed herein are directed to a non-invasive way to collect cervical-vaginal fluids and isolate genetic material (e.g., exosome or microvesicle mRNA, in several embodiments). Components of cervical-vaginal fluid are thought to be complex and may include mucus, cells, and plasma transudate with inorganic salts, urea, amino acids, proteins and volatile fatty acids, among other compositions. Cervical-vaginal fluid exosomes and microvesicles may provide a novel and highly concentrated source of biomarkers for detecting early signs of gynecological diseases and conditions. Exosomes and microvesicles are nm-sized particles that are shed from all cell types into biofluids such as plasma, urine, saliva, ascites, etc. They contain proteins and nucleic acids such as miRNA and mRNA which are representative of the cells in which they are derived from. For example, nucleic acids can be associated with one or more different types of membrane particles (ranging in size from 50-80 nm), exosomes (ranging in size from 50-100 nm), exosome-like vesicles (ranging in size from 20-50 nm), and microvesicles (ranging in size from 100-1000 nm). In several embodiments, these vesicles are isolated and/or concentrated, thereby preserving vesicle associated RNA even if there is a high RNAse extracellular environment. The RNAs within these particles have been shown to be functional and can confer specific activity to target cells.

Exosomes and microvesicles originating from the female reproductive tract have been isolated from normal and diseased conditions. Several studies have examined protein and microRNA profiles from ovarian cancer cell- and ascites-derived exosomes. The miRNA and protein profiles from tumor-derived exosomes are significantly different than those derived from benign cases. Thus, as described in several embodiments herein, exosomes and microvesicles and their contents can be used as diagnostic markers for screening, detecting and/or monitoring (or other treatment) of gynecological diseases and conditions.

In several embodiments of the methods disclosed herein, cervical-vaginal fluids are collected using a tampon. Tampons are made of cotton and/or rayon fibers and typically include a cotton cord and applicator. There are several types of tampons categorized by the amount of fluid it can absorb. The industry standards are light (absorbs up to 6 g), regular (absorbs 6-9 g), and super (absorbs (9-12 g)). The amount of cervical-vaginal fluid produced varies depending on the individual and throughout the menstrual cycle. The amount depends on hormone levels in the body but the average produced is 1.55+/−0.6 g per 8 hours. Therefore, cervical-vaginal fluids can be collected using the light type tampon worn for about 1 hour to about 8 hours, including about 2 hours to about 7 hours, about 3 hours to about 6 hours, about 4 hours to about 5 hours, or any time period in between. Following the time period, the tampon is removed and can be placed at about 4° C. for about 1 hour to about 4 hours. For example, the tampon can be placed at about 4° C. for about 1 to about 2 hours, about 2 to about 3 hours, about 3 hours to about 4 hours or any time period in between. The tampon may be incubated at temperatures ranging from about 0° C. to about 10° C., depending on the embodiment. For example, in several embodiments the tampon may be incubated at a temperature between about 0° C. to about 2° C., about 2° C. to about 4° C., about 4° C. to about 6° C., about 6° C. to about 8° C., about 8° C. to about 10° C., and any temperature therein. The tampon can then be stored at about −80° C. until further processing. Other low temperatures may also be used, depending on the embodiment. For example, the tampon can be stored at about −20° C. to about −100° C., including about −20° C. to about −40° C., including about −40° C. to about −60° C., about −60° C. to about −80° C., about −80° C. to about −100° C., and temperatures therebetween. In some embodiments, mRNA profiles of the exosomes and microvesicles are found to be stable in these conditions when compared to immediate processing.

A variety of methods can be used, according to the embodiments disclosed herein, to efficiently capture and preserve vesicle associated RNA. In several embodiments, centrifugation on a density gradient to fractionate the non-cellular portion of the sample is performed. In some embodiments, density centrifugation is optionally followed by high speed centrifugation to cause vesicle sedimentation or pelleting. As such approaches may be time consuming and may require expensive and specialized equipment in several embodiments, low speed centrifugation can be employed to collect vesicles.

In several embodiments, filtration (alone or in combination with centrifugation) is used to capture vesicles of different sizes. In some embodiments, differential capture of vesicles is made based on the surface expression of protein markers. For example, a filter may be designed to be reactive to a specific surface marker (e.g., filter coupled to an antibody) or specific types of vesicles or vesicles of different origin. In several embodiments, the combination of filtration and centrifugation allows a higher yield or improved purity of vesicles.

In some embodiments, the markers are unique vesicle proteins or peptides. In some embodiments, the severity of a particular gynecological disease or disorder is associated with certain vesicle modifications which can be exploited to allow isolation of particular vesicles. Modification may include, but is not limited to addition of lipids, carbohydrates, and other molecules such as acylated, formylated, lipoylated, myristolylated, palmitoylated, alkylated, methylated, isoprenylated, prenylated, amidated, glycosylated, hydroxylated, iodinated, adenylated, phosphorylated, sulfated, and selenoylated, ubiquitinated. In some embodiments, the vesicle markers comprise non-proteins such as lipids, carbohydrates, nucleic acids, RNA, DNA, etc.

In several embodiments, the specific capture of vesicles based on their surface markers also enables a “dip stick” format where each different type of vesicle is captured by dipping probes coated with different capture molecules (e.g., antibodies with different specificities) into a patient sample.

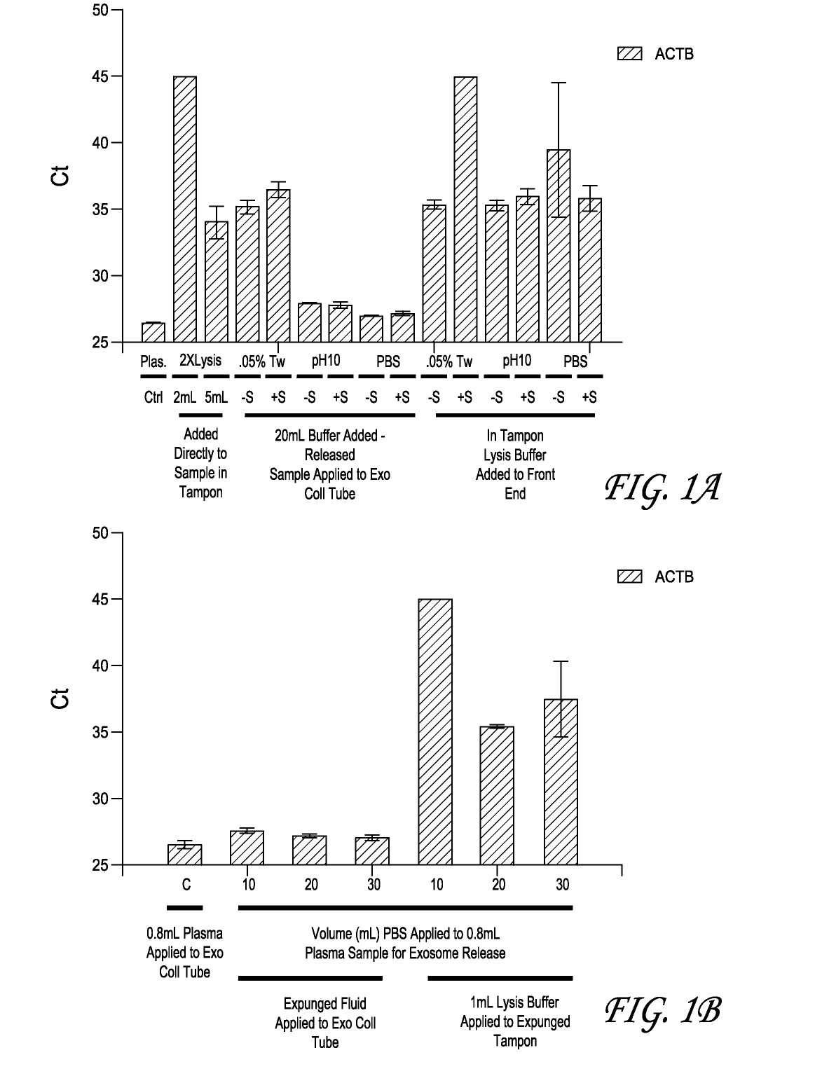

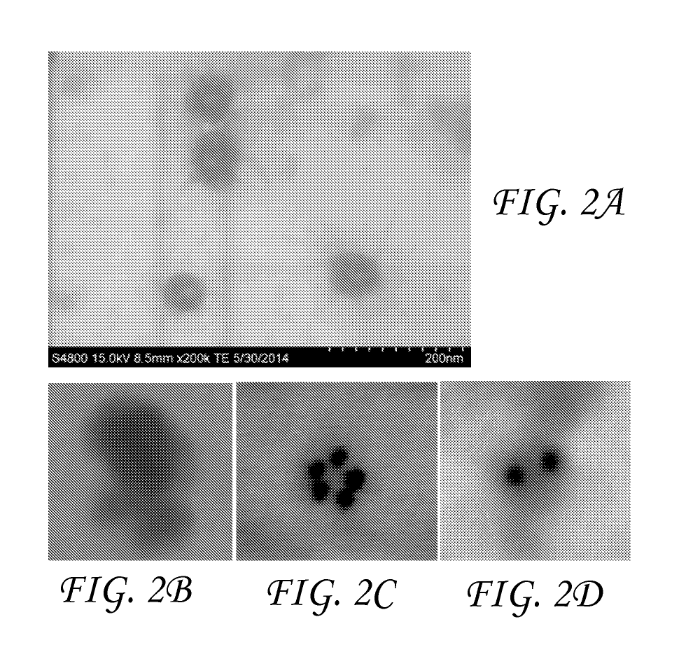

In some embodiments, tampons may be used to collect cervical-vaginal fluid cells, exosomes and microvesicles and keep them intact at least for 8 hours while being worn. In some embodiments, once the cervical-vaginal fluid is absorbed into the tampon, the cells, exosomes and microvesicles can be released when a large amount of non-detergent containing buffer such as PBS, is applied and then expunged using a syringe or similar device large enough to accommodate the size of the tampon. Volumes of buffer equal to or greater than 10 mL may be sufficient to release cells, exosomes and microvesicles from a light type tampon (FIG. 1B). In some embodiments, after expunging the fluids, a low speed centrifugation step may be used to separate any cells or debris. If analysis of the cell components is desired, in some embodiments the supernatant can be discarded and the cell pellet can be retained. If analysis of smaller membrane particles such as exosomes and microvesicles is desired, in some embodiments the supernatant from this low speed spin can be the starting point for further isolation using conventional ultracentrifugation or can then be added to an exosome and microvesicle-capture filter device. In some embodiments, after application of the supernatant to the filter device, another low speed spin may be used to concentrate the particles onto the filter and remove the liquid. A lysis buffer may be added to the filter to release RNA. A low speed spin may be used to transfer the lysate from the filter device and in to the wells of an oligo(dT)-coated plate. The mRNA from the sample may be hybridized to the plate and the captured mRNA can be eluted and may be used for further downstream analysis. In several embodiments, after collection of the cervical-vaginal fluid containing membrane particles, cells, exosomes and microvesicles, molecular analysis of DNA, protein, membrane surface antigens, and miRNA can be performed in addition to mRNA analysis.

### Methodology

Free extracellular RNA is quickly degraded by nucleases, making it a potentially poor diagnostic marker. As described above, some extracellular RNA is associated with particles or vesicles that can be found in various biological samples, such as cervical-vaginal fluid. This vesicle associated RNA, which includes mRNA, is protected from the degradation processes in the cervical-vaginal fluid. Microvesicles are shed from most cell types and consist of fragments of plasma membrane. Microvesicles contain RNA, mRNA, microRNA, and proteins and mirror the composition of the cell from which they are shed. Exosomes are small microvesicles secreted by a wide range of mammalian cells and are secreted under normal and pathological conditions. These vesicles contain certain proteins and RNA including mRNA and microRNA. Several embodiments evaluate nucleic acids such as small interfering RNA (siRNA), tRNA, and small activating RNA (saRNA), among others.

In several embodiments the RNA isolated from vesicles from the cervical-vaginal fluid of a patient is used as a template to make complementary DNA (cDNA), for example through the use of a reverse transcriptase. In several embodiments, cDNA is amplified using the polymerase chain reaction (PCR). In other embodiments, amplification of nucleic acid and RNA may also be achieved by any suitable amplification technique such as nucleic acid based amplification (NASBA) or primer-dependent continuous amplification of nucleic acid, or ligase chain reaction. Other methods may also be used to quantify the nucleic acids, such as for example, including Northern blot analysis, RNAse protection assay, RNA sequencing, RT-PCR, real-time RT-PCR, nucleic acid sequence-based amplification, branched-DNA amplification, ELISA, mass spectrometry, CHIP-sequencing, and DNA or RNA microarray analysis.

In several embodiments, mRNA is quantified by a method entailing cDNA synthesis from mRNA and amplification of cDNA using PCR. In one preferred embodiment, a multi-well filterplate is washed with lysis buffer and wash buffer. A cDNA synthesis buffer is then added to the multi-well filterplate. The multi-well filterplate can be centrifuged. PCR primers are added to a PCR plate, and the cDNA is transferred from the multi-well filterplate to the PCR plate. The PCR plate is centrifuged, and real time PCR is commenced.

Another preferred embodiment comprises application of specific antisense primers during mRNA hybridization or during cDNA synthesis. It is preferable that the primers be added during mRNA hybridization, so that excess antisense primers may be removed before cDNA synthesis to avoid carryover effects. The oligo(dT) and the specific primer (NNNN) simultaneously prime cDNA synthesis at different locations on the poly-A RNA. The specific primer (NNNN) and oligo(dT) cause the formation of cDNA during amplification. Even when the specific primer-derived cDNA is removed from the GenePlate by heating each well, the amounts of specific cDNA obtained from the heat denaturing process (for example, using TaqMan quantitative PCR) is similar to the amount obtained from an un-heated negative control. This allows the heat denaturing process to be completely eliminated. Moreover, by adding multiple antisense primers for different targets, multiple genes can be amplified from the aliquot of cDNA, and oligo(dT)-derived cDNA in the GenePlate can be stored for future use.

Another alternative embodiment involves a device for high-throughput quantification of mRNA from cervical-vaginal fluid. The device includes a multi-well filterplate containing: multiple sample-delivery wells, an exosome-capturing filter (or filter directed to another biological component of interest) underneath the sample-delivery wells, and an mRNA capture zone under the filter, which contains oligo(dT)-immobilized in the wells of the mRNA capture zone. In order to increase the efficiency of exosome collection, several filtration membranes can be layered together.

In some embodiments, amplification comprises conducting real-time quantitative PCR (TaqMan) with exosome-derived RNA and control RNA. In some embodiments, a Taqman assay is employed. The 5′ to 3′ exonuclease activity of Taq polymerase is employed in a polymerase chain reaction product detection system to generate a specific detectable signal concomitantly with amplification. An oligonucleotide probe, nonextendable at the 3′ end, labeled at the 5′ end, and designed to hybridize within the target sequence, is introduced into the polymerase chain reaction assay. Annealing of the probe to one of the polymerase chain reaction product strands during the course of amplification generates a substrate suitable for exonuclease activity. During amplification, the 5′ to 3′ exonuclease activity of Taq polymerase degrades the probe into smaller fragments that can be differentiated from undegraded probe. In other embodiments, the method comprises: (a) providing to a PCR assay containing a sample, at least one labeled oligonucleotide containing a sequence complementary to a region of the target nucleic acid, wherein the labeled oligonucleotide anneals within the target nucleic acid sequence bounded by the oligonucleotide primers of step (b); (b) providing a set of oligonucleotide primers, wherein a first primer contains a sequence complementary to a region in one strand of the target nucleic acid sequence and primes the synthesis of a complementary DNA strand, and a second primer contains a sequence complementary to a region in a second strand of the target nucleic acid sequence and primes the synthesis of a complementary DNA strand; and wherein each oligonucleotide primer is selected to anneal to its complementary template upstream of any labeled oligonucleotide annealed to the same nucleic acid strand; (c) amplifying the target nucleic acid sequence employing a nucleic acid polymerase having 5′ to 3′ nuclease activity as a template dependent polymerizing agent under conditions which are permissive for PCR cycling steps of (i) annealing of primers and labeled oligonucleotide to a template nucleic acid sequence contained within the target region, and (ii) extending the primer, wherein said nucleic acid polymerase synthesizes a primer extension product while the 5′ to 3′ nuclease activity of the nucleic acid polymerase simultaneously releases labeled fragments from the annealed duplexes comprising labeled oligonucleotide and its complementary template nucleic acid sequences, thereby creating detectable labeled fragments; and (d) detecting and/or measuring the release of labeled fragments to determine the presence or absence of target sequence in the sample.

In alternative embodiments, a Taqman assay is employed that provides a reaction that results in the cleavage of single-stranded oligonucleotide probes labeled with a light-emitting label wherein the reaction is carried out in the presence of a DNA binding compound that interacts with the label to modify the light emission of the label. The method utilizes the change in light emission of the labeled probe that results from degradation of the probe. The methods are applicable in general to assays that utilize a reaction that results in cleavage of oligonucleotide probes, and in particular, to homogeneous amplification/detection assays where hybridized probe is cleaved concomitant with primer extension. A homogeneous amplification/detection assay is provided which allows the simultaneous detection of the accumulation of amplified target and the sequence-specific detection of the target sequence.

In alternative embodiments, real-time PCR formats may also be employed. One format employs an intercalating dye, such as SYBR Green. This dye provides a strong fluorescent signal on binding double-stranded DNA; this signal enables quantification of the amplified DNA. Although this format does not permit sequence-specific monitoring of amplification, it enables direct quantization of amplified DNA without any labeled probes. Other such fluorescent dyes that may also be employed are SYBR Gold, YO-PRO dyes and Yo Yo dyes.

Another real-time PCR format that may be employed uses reporter probes that hybridize to amplicons to generate a fluorescent signal. The hybridization events either separate the reporter and quencher moieties on the probes or bring them into closer proximity. The probes themselves are not degraded and the reporter fluorescent signal itself is not accumulated in the reaction. The accumulation of products during PCR is monitored by an increase in reporter fluorescent signal when probes hybridize to amplicons. Formats in this category include molecular beacons, dual-hybe probes, Sunrise or Amplifluor, and Scorpion real-time PCR assays.

Another real-time PCR format that may also be employed is the so-called “Policeman” system. In this system, the primer comprises a fluorescent moiety, such as FAM, and a quencher moiety which is capable of quenching fluorescence of the fluorescent moiety, such as TAMRA, which is covalently bound to at least one nucleotide base at the 3′ end of the primer. At the 3′ end, the primer has at least one mismatched base and thus does not complement the nucleic acid sample at that base or bases. The template nucleic acid sequence is amplified by PCR with a polymerase having 3′-5′ exonuclease activity, such as the Pfu enzyme, to produce a PCR product. The mismatched base(s) bound to the quencher moiety are cleaved from the 3′ end of the PCR product by 3′-5′ exonuclease activity. The fluorescence that results when the mismatched base with the covalently bound quencher moiety is cleaved by the polymerase, thus removing the quenching effect on the fluorescent moiety, is detected and/or quantified at least one time point during PCR. Fluorescence above background indicates the presence of the synthesized nucleic acid sample.

Another alternative embodiment involves a fully automated system for performing high throughput quantification of mRNA in cervical-vaginal fluid, including: robots to apply cervical-vaginal fluid samples, hypotonic buffer, and lysis buffer to the device; an automated vacuum aspirator and centrifuge, and automated PCR machinery.

The method of determining the presence of a gynecological disease or condition disclosed may also employ other methods of measuring mRNA other than those described above. Other methods which may be employed include, for example, Northern blot analysis, Rnase protection, solution hybridization methods, semi-quantitative RT-PCR, and in situ hybridization.

In some embodiments, in order to properly quantify the amount of mRNA, quantification is calculated by comparing the amount of mRNA encoding a marker of gynecological disease or condition to a reference value. In some embodiments the reference value will be the amount of mRNA found in healthy non-diseased patients. In other embodiments, the reference value is the expression level of a house-keeping gene. In certain such embodiments, beta-actin, or other appropriate housekeeping gene is used as the reference value. Numerous other house-keeping genes that are well known in the art may also be used as a reference value. In other embodiments, a house keeping gene is used as a correction factor, such that the ultimate comparison is the expression level of marker from a diseased patient as compared to the same marker from a non-diseased (control) sample. In several embodiments, the house keeping gene is a tissue specific gene or marker, such as those discussed above. In still other embodiments, the reference value is zero, such that the quantification of the markers is represented by an absolute number. In several embodiments a ratio comparing the expression of one or more markers from a diseased patient to one or more other markers from a non-diseased person is made. In several embodiments, the comparison to the reference value is performed in real-time, such that it may be possible to make a determination about the sample at an early stage in the expression analysis. For example, if a sample is processed and compared to a reference value in real time, it may be determined that the expression of the marker exceeds the reference value after only a few amplification cycles, rather than requiring a full-length analysis. In several embodiments, this early comparison is particularly valuable, such as when a rapid diagnosis and treatment plan are required (e.g., to treat aggressive cancers or infections prior to possible development of sepsis).

In alternative embodiments, the ability to determine the total efficiency of a given sample by using known amounts of spiked standard RNA results from embodiments being dose-independent and sequence-independent. The use of known amounts of control RNA allows PCR measurements to be converted into the quantity of target mRNAs in the original samples.

In some embodiments, a kit is provided for extracting target components from fluid sample. In some embodiments, a kit comprises a capture device and additional items useful to carry out methods disclosed herein. In some embodiments, a kit comprises one or more reagents selected from the group consisting of lysis buffers, chaotropic reagents, washing buffers, alcohol, detergent, or combinations thereof. In some embodiments, kit reagents are provided individually or in storage containers. In several embodiments, kit reagents are provided ready-to-use. In some embodiments, kit reagents are provided in the form of stock solutions that are diluted before use. In some embodiments, a kit comprises plastic parts (optionally sterilized or sterilizable) that are useful to carry out methods herein disclosed. In some embodiments, a kit comprises plastic parts selected from the group consisting of racks, centrifuge tubes, vacuum manifolds, and multi-well plates. Instructions for use are also provided, in several embodiments.

In some embodiments, a specialized feminine hygiene product is provided to a patient for use in collecting cervical-vaginal fluids. In some embodiments, the specialized product is designed to fit with a cervical-vaginal fluid collection system for the quick processing and isolation of a biological component of interest after the product is used by a patient. For instance, in some embodiments, the shape of the feminine hygiene product may allow it to be placed into either a specialized or over-the-counter syringe that can remove biological materials that were collected by the hygiene product after it has been used by a patient. In other embodiments, the hygiene product may be compatible with a filter device. The product may slide or lock into place within the filter device so that buffer solution or some other composition can be used to release the collected biological materials straight from the hygiene product onto a filter material within the device. In some embodiments, the specialized product is comprised of a material that allows it to better absorb certain biological components. For instance, certain fabrics may have more absorptive properties with respect to certain biological components and could be used for sample collection. Alternatively, chemical compositions included in the hygiene product could aid in either the release of the collected biological sample from the product or otherwise play a role in sample processing. In other embodiments, just a subset of the feminine hygiene product may be isolated or removed for the rest of the product and used for sampling. For instance, a portion of a sanitary pad may be torn or peeled away from the rest of the pad material and then inserted into a syringe or filter device. Instructions with a specialized hygiene product might direct a patient to place the product (or a subset of the product) in a specialized or over-the-counter container and send it to a laboratory for testing. Alternatively, the design may aid in the ability of at-home testing methods that may employ other equipment that can process a sample without the need to send the sample to a lab or hospital facility.

In several embodiments, the analyses described herein are applicable to human patients, while in some embodiments, the methods are applicable to animals (e.g., veterinary diagnoses).

In several embodiments, presence of a gynecological condition or disease induces the expression of one or more markers. In several embodiments, the increased expression is measured by the amount of mRNA encoding said markers (in other embodiments, DNA or protein are used to measure expression levels). In some embodiments cervical-vaginal fluid is collected from a patient and directly evaluated. In some embodiments, vesicles are concentrated, for example by use of filtration or centrifugation. Isolated vesicles are then incubated with lysis buffer to release the RNA from the vesicles, the RNA then serving as a template for cDNA which is quantified with methods such as quantitative PCR (or other appropriate amplification or quantification technique). In several embodiments, the level of specific marker RNA from patient vesicles is compared with a desired control such as, for example, RNA levels from a healthy patient population, or the RNA level from an earlier time point from the same patient or a control gene from the same patient.

### Implementation Mechanisms

According to some embodiments, the methods described herein can be implemented by one or more special-purpose computing devices. The special-purpose computing devices may be hard-wired to perform the techniques, or may include digital electronic devices such as one or more application-specific integrated circuits (ASICs) or field programmable gate arrays (FPGAs) that are persistently programmed to perform the techniques, or may include one or more general purpose hardware processors programmed to perform the techniques pursuant to program instructions in firmware, memory, other storage, or a combination. Such special-purpose computing devices may also combine custom hard-wired logic, ASICs, or FPGAs with custom programming to accomplish the techniques. The special-purpose computing devices may be desktop computer systems, server computer systems, portable computer systems, handheld devices, networking devices or any other device or combination of devices that incorporate hard-wired and/or program logic to implement the techniques.

Computing device(s) are generally controlled and coordinated by operating system software, such as iOS, Android, Chrome OS, Windows XP, Windows Vista, Windows 7, Windows 8, Windows Server, Windows CE, Unix, Linux, SunOS, Solaris, iOS, Blackberry OS, VxWorks, or other compatible operating systems. In other embodiments, the computing device may be controlled by a proprietary operating system. Conventional operating systems control and schedule computer processes for execution, perform memory management, provide file system, networking, I/O services, and provide a user interface functionality, such as a graphical user interface (“GUI”), among other things.

In some embodiments, the computer system includes a bus or other communication mechanism for communicating information, and a hardware processor, or multiple processors, coupled with the bus for processing information. Hardware processor(s) may be, for example, one or more general purpose microprocessors.

In some embodiments, the computer system may also includes a main memory, such as a random access memory (RAM), cache and/or other dynamic storage devices, coupled to a bus for storing information and instructions to be executed by a processor. Main memory also may be used for storing temporary variables or other intermediate information during execution of instructions to be executed by the processor. Such instructions, when stored in storage media accessible to the processor, render the computer system into a special-purpose machine that is customized to perform the operations specified in the instructions.

In some embodiments, the computer system further includes a read only memory (ROM) or other static storage device coupled to bus for storing static information and instructions for the processor. A storage device, such as a magnetic disk, optical disk, or USB thumb drive (Flash drive), etc., may be provided and coupled to the bus for storing information and instructions.

In some embodiments, the computer system may be coupled via a bus to a display, such as a cathode ray tube (CRT) or LCD display (or touch screen), for displaying information to a computer user. An input device, including alphanumeric and other keys, is coupled to the bus for communicating information and command selections to the processor. Another type of user input device is cursor control, such as a mouse, a trackball, or cursor direction keys for communicating direction information and command selections to the processor and for controlling cursor movement on display. This input device typically has two degrees of freedom in two axes, a first axis (e.g., x) and a second axis (e.g., y), that allows the device to specify positions in a plane. In some embodiments, the same direction information and command selections as cursor control may be implemented via receiving touches on a touch screen without a cursor.

In some embodiments, the computing system may include a user interface module to implement a GUI that may be stored in a mass storage device as executable software codes that are executed by the computing device(s). This and other modules may include, by way of example, components, such as software components, object-oriented software components, class components and task components, processes, functions, attributes, procedures, subroutines, segments of program code, drivers, firmware, microcode, circuitry, data, databases, data structures, tables, arrays, and variables.

In general, the word “module,” as used herein, refers to logic embodied in hardware or firmware, or to a collection of software instructions, possibly having entry and exit points, written in a programming language, such as, for example, Java, Lua, C or C++. A software module may be compiled and linked into an executable program, installed in a dynamic link library, or may be written in an interpreted programming language such as, for example, BASIC, Perl, or Python. It will be appreciated that software modules may be callable from other modules or from themselves, and/or may be invoked in response to detected events or interrupts. Software modules configured for execution on computing devices may be provided on a computer readable medium, such as a compact disc, digital video disc, flash drive, magnetic disc, or any other tangible medium, or as a digital download (and may be originally stored in a compressed or installable format that requires installation, decompression or decryption prior to execution). Such software code may be stored, partially or fully, on a memory device of the executing computing device, for execution by the computing device. Software instructions may be embedded in firmware, such as an EPROM. It will be further appreciated that hardware modules may be comprised of connected logic units, such as gates and flip-flops, and/or may be comprised of programmable units, such as programmable gate arrays or processors. The modules or computing device functionality described herein are preferably implemented as software modules, but may be represented in hardware or firmware. Generally, the modules described herein refer to logical modules that may be combined with other modules or divided into sub-modules despite their physical organization or storage

In some embodiments, a computer system may implement the methods described herein using customized hard-wired logic, one or more ASICs or FPGAs, firmware and/or program logic which in combination with the computer system causes or programs the computer system to be a special-purpose machine. According to one embodiment, the methods herein are performed by the computer system in response to hardware processor(s) executing one or more sequences of one or more instructions contained in main memory. Such instructions may be read into main memory from another storage medium, such as a storage device. Execution of the sequences of instructions contained in main memory causes processor(s) to perform the process steps described herein. In alternative embodiments, hard-wired circuitry may be used in place of or in combination with software instructions.

The term “non-transitory media,” and similar terms, as used herein refers to any media that store data and/or instructions that cause a machine to operate in a specific fashion. Such non-transitory media may comprise non-volatile media and/or volatile media. Non-volatile media includes, for example, optical or magnetic disks, or other types of storage devices. Volatile media includes dynamic memory, such as a main memory. Common forms of non-transitory media include, for example, a floppy disk, a flexible disk, hard disk, solid state drive, magnetic tape, or any other magnetic data storage medium, a CD-ROM, any other optical data storage medium, any physical medium with patterns of holes, a RAM, a PROM, and EPROM, a FLASH-EPROM, NVRAM, any other memory chip or cartridge, and networked versions of the same.

Non-transitory media is distinct from but may be used in conjunction with transmission media. Transmission media participates in transferring information between nontransitory media. For example, transmission media includes coaxial cables, copper wire and fiber optics, including the wires that comprise a bus. Transmission media can also take the form of acoustic or light waves, such as those generated during radio-wave and infra-red data communications.

Various forms of media may be involved in carrying one or more sequences of one or more instructions to a processor for execution. For example, the instructions may initially be carried on a magnetic disk or solid state drive of a remote computer. The remote computer can load the instructions into its dynamic memory and send the instructions over a telephone line using a modem or other network interface, such as a WAN or LAN interface. A modem local to a computer system can receive the data on the telephone line and use an infra-red transmitter to convert the data to an infra-red signal. An infra-red detector can receive the data carried in the infra-red signal and appropriate circuitry can place the data on a bus. The bus carries the data to the main memory, from which the processor retrieves and executes the instructions. The instructions received by the main memory may retrieve and execute the instructions. The instructions received by the main memory may optionally be stored on a storage device either before or after execution by the processor.

In some embodiments, the computer system may also include a communication interface coupled to a bus. The communication interface may provide a two-way data communication coupling to a network link that is connected to a local network. For example, a communication interface may be an integrated services digital network (ISDN) card, cable modem, satellite modem, or a modem to provide a data communication connection to a corresponding type of telephone line. As another example, a communication interface may be a local area network (LAN) card to provide a data communication connection to a compatible LAN (or WAN component to communicate with a WAN). Wireless links may also be implemented. In any such implementation, a communication interface sends and receives electrical, electromagnetic or optical signals that carry digital data streams representing various types of information.

A network link may typically provide data communication through one or more networks to other data devices. For example, a network link may provide a connection through a local network to a host computer or to data equipment operated by an Internet Service Provider (ISP). The ISP in turn provides data communication services through the world wide packet data communication network now commonly referred to as the “Internet.” The local network and Internet both use electrical, electromagnetic or optical signals that carry digital data streams. The signals through the various networks and the signals on the network link and through a communication interface, which carry the digital data to and from the computer system, are example forms of transmission media.

In some embodiments, the computer system can send messages and receive data, including program code, through the network(s), the network link, and the communication interface. In the Internet example, a server might transmit a requested code for an application program through the Internet, ISP, local network, and communication interface.

The received code may be executed by a processor as it is received, and/or stored in a storage device, or other non-volatile storage for later execution.

### EXAMPLES

In some embodiments, cervical-vaginal fluids were collected by a tampon, and exosomes and microvesicles were released from the interwoven fibers with the addition of excess buffer. Depending on the embodiments, exosomes and microvesicles may have an average size of approximately 186 nm and be CD9- and CD63-positive based on immunogold scanning electron microscopy. In a single isolation protocol, several embodiments allowed collection of up to ˜2×10¹⁵ exosomes and microvesicles. To concentrate the exosomes and microvesicles and isolate mRNA, in some embodiments a filter device specifically designed to handle exosomes and microvesicles in large volumes was employed. In several embodiments, the exosomes and microvesicle RNAs were released by a lysis buffer and hybridized to an oligo(dT)-coated plate. In these embodiments, the eluted poly(A)+RNA could be used as starting material for RNA sequencing library preparation. In other embodiments, the exosomes and microvesicle RNAs were released and hybridized to a T7 promoter oligo(dT)-coated plate. In-plate RNA amplification synthesizes additional quantities of RNA which is used as starting material for RNA sequencing library preparation. RNA sequencing data analyses from these methods indicated that major functional annotation clusters involved keratinocyte and cell differentiation, small proline rich proteins, and antigen processing and presentation. Among the most highly expressed genes in some embodiments were IL-8, FTL, B2M, S100A8, S100A9, SAT1, IFITM2, SPRR3, SOD2, FTH1, CXCL8, G0S2, SRGN, IL-1B, and CXCL1. The collection method and streamlined platform for vaginal fluid exosome molecular analysis allow a simple method of examining the role of exosomes and microvesicles in ovarian-, uterus- and cervical-related diseases and conditions.

### Example 1. Various Conditions and Buffers were Attempted to Determine the Optimal Method of Cervical Vaginal Fluid Exosome and Microvesicle Release. ACTB Expression Level was Used as a Reference Gene to Compare the Different Methods

In the example in FIG. 1A, 0.8 mL plasma in 2 mL PBS was applied to each tampon. Plasma contains exosomes and microvesicles and was used as a test sample to determine proof-of-concept. The plasma sample was allowed to absorb into the tampon for 5 minutes. In other embodiments, the plasma sample may absorb into the tampon for about 1 minute to about 30 minutes, 1 minute to 25 minutes, 1 minute to 20 minutes, 1 minute to 15 minutes, 1 minute to 10 minutes, 1 minute to 5 minutes, 1 minute to 4 minutes, 1 minute to 3 minutes, 1 minute to 2 minutes, or any time period in between. Various methods were then applied to the tampons for testing. For example, in one instance, 2 or 5 mL 2× lysis buffer was applied directly to the plasma-absorbed tampon. In other embodiments, 2× lysis buffer can be applied in the amount of about 1 mL to about 10 mL, about 1 mL to about 9 mL, about 1 mL to about 8 mL, about 1 mL to about 7 mL, about 1 mL to about 6 mL, about 1 mL to about 5 mL, about 1 mL to about 4 mL, about 1 mL to about 3 mL, about 1 mL to about 2 mL, and any amount in between. After a 10-minute incubation at 37° C., the lysate was removed from the tampon using a syringe. In other embodiments, incubation may be from about 5 minutes to about 30 minutes, about 5 minutes to about 25 minutes, about 5 minutes to about 20 minutes, about 5 minutes to about 15 minutes, about 5 minutes to about 10 minutes, or any time in between. In other embodiments, incubation may be performed at about 30° C. to about 45° C., about 30° C. to about 44° C., about 30° C. to about 43° C., about 30° C. to about 42° C., about 30° C. to about 41° C., about 30° C. to about 40° C., about 30° C. to about 39° C., about 30° C. to about 38° C., about 30° C. to about 37° C., or any time in between. This lysate which may contain cell, exosome and microvesicle components was applied directly to the oligo(dT)-coated plate. In other cases, about 5 mL to about 10 mL, about 10 mL to about 15 mL, about 15 mL to about 20 mL, or any range in between of various buffers such as 0.05% tween, PBS, or 0.32M NaHCO3, pH10 can be applied to the plasma-absorbed tampon, allowed to incubate for 5 minutes, and treated with or without a 15 s sonication. A 20 cc syringe was used to expunge the excess fluid from the tampon. This fluid was applied to an exosome and microvesicle capture filter device. The capture filter device can accommodate volumes up to 12.5 mL so an additional spin was necessary to filter the entire volume. In other embodiments, the capture filter device can accommodate about 5 mL to about 10 mL, about 10 mL to about 15 mL, about 15 mL to about 20 mL, or any volume in between. After two 15 minute spins at 800×g, the tip of the filter device was removed and placed on a holder above a 96-well plate. In other embodiments, the filter device is spun for about 5 minutes to about 10 minutes, about 10 minutes to about 15 minutes, about 15 minutes to about 20 minutes, or any time in between. In other embodiments, only a single spin is performed. In other embodiments, the spin is at about 200×g to about 400×g, about 400×g to about 600×g, about 600×g to about 800×g, about 800×g to about 1,000×g, or any speed in between. The tip encases the filter which has the exosome and microvesicles captured on the membranes. lx lysis buffer is applied to the filter and incubated for 10 minutes at 37° C. In other embodiments, incubation may be from about 5 minutes to about 30 minutes, about 5 minutes to about 25 minutes, about 5 minutes to about 20 minutes, about 5 minutes to about 15 minutes, about 5 minutes to about 10 minutes, or any time in between. In other embodiments, incubation may be performed at about 30° C. to about 45° C., about 30° C. to about 44° C., about 30° C. to about 43° C., about 30° C. to about 42° C., about 30° C. to about 41° C., about 30° C. to about 40° C., about 30° C. to about 39° C., about 30° C. to about 38° C., about 30° C. to about 37° C., or any time in between. Following the incubation, the tip and holder is placed above an oligo(dT)-coated plate and a 5 minute spin at 2000×g transfers the lysate from the filter tip to the well of the plate. In other embodiments, the spin is performed for about 5 minutes to about 10 minutes, about 10 minutes to about 15 minutes, about 15 minutes to about 20 minutes, or any time in between. In other embodiments, the spin is at about 200×g to about 400×g, about 400×g to about 600×g, about 600×g to about 800×g, about 800×g to about 1,000×g, or any speed in between. Samples were hybridized overnight at around 4° C. In other embodiments, the samples can be hybridized at about 1° C. to about 2° C., about 2° C. to about 3° C., about 3° C. to about 4° C., about 4° C. to about 5° C., about 6° C. to about 7° C., about 7° C. to about 8° C., or any temperature in between. To confirm that most cells, exosomes and microvesicles were removed from the tampon in the step before, 1 mL 1× lysis buffer was added to the expunged tampon. The tampon was incubated at 37° C. for 10 minutes and expunged using the syringe method. In other embodiments, incubation may be from about 5 minutes to about 30 minutes, about 5 minutes to about 25 minutes, about 5 minutes to about 20 minutes, about 5 minutes to about 15 minutes, about 5 minutes to about 10 minutes, or any time in between. In other embodiments, incubation may be performed at about 30° C. to about 45° C., about 30° C. to about 44° C., about 30° C. to about 43° C., about 30° C. to about 42° C., about 30° C. to about 41° C., about 30° C. to about 40° C., about 30° C. to about 39° C., about 30° C. to about 38° C., about 30° C. to about 37° C., or any time in between. This lysate was then applied to the oligo(dT)-coated plate and hybridized in a similar manner as above. For the control sample, 0.8 mL plasma in 2 mL PBS was applied directly to the exosome and microvesicle capture filter device. All steps for this sample are the same as those described above following the application of the fluids to the device.

Following mRNA hybridization at 4° C. overnight, the wells of the oligo(dT)-coated plate were washed with Wash Buffer A and B three times each. After removal of all traces of Wash Buffer B, 30 uL cDNA master mix with random primers was added to each well and incubated at 37° C. for 1 hour. In other embodiments, incubation may be performed at about 30° C. to about 45° C., about 30° C. to about 44° C., about 30° C. to about 43° C., about 30° C. to about 42° C., about 30° C. to about 41° C., about 30° C. to about 40° C., about 30° C. to about 39° C., about 30° C. to about 38° C., about 30° C. to about 37° C., or any time in between. In other embodiments, inclubation can be performed for about 30 minutes to about 1 hour, about 1 hour to about 2 hours, about 2 hours to about 3 hours, about 3 hours to about 4 hours, about 4 hours to about 5 hours, about 5 hours to about 6 hours, about 6 hours to about 7 hours, about 7 hours to about 8 hours, or any time in between. Two μL of cDNA reactions were added to Sso Advance Universal (Bio-Rad) reaction mix with ACTB primers. The qPCR reaction was performed under standard conditions using ABI ViiA7 instrument and software.

To determine the optimal volume required to remove exosomes and microvesicles from the tampon, volumes of PBS from 10-30 mL were tested for their efficacy. A positive control of 0.8 mL plasma in 2 mL PBS applied directly to the exosome and microvesicle capture filter device was used as in the previous example. In each of the other samples, 0.8 mL plasma in 2 mL PBS was applied to the tampon and incubated for 5 minutes at room temperature to allow complete absorption. In other embodiments, PBS can be applied and incubated for about 1 minute to about 30 minutes, about 1 minute to about 25 minutes, about 1 minute to about 20 minutes, about 1 minute to about 15 minutes, about 1 minute to about 10 minutes, about 1 minute to about 5 minutes, about 1 minute to about 4 minutes, about 1 minute to about 3 minutes, about 1 minute to about 2 minutes, or any time period in between. PBS in the volumes of 10, 20 and 30 mL were applied to the tampons and incubated for 5 minutes to allow for complete release from the fibers. In other embodiments, PBS can be applied and incubated for about 1 minute to about 30 minutes, about 1 minute to about 25 minutes, about 1 minute to about 20 minutes, about 1 minute to about 15 minutes, about 1 minute to about 10 minutes, about 1 minute to about 5 minutes, about 1 minute to about 4 minutes, about 1 minute to about 3 minutes, about 1 minute to about 2 minutes, or any time period in between. The fluid was then expunged from the tampon using a 20 cc syringe. In other embodiments, the fluid can be expunged using a about 5 cc to about 10 cc syringe, about 10 cc to about 20 cc syringe, about 20 cc to about 50 cc syringe, or about 50 cc to about 100 cc syringe. The excess fluid was applied to an exosome and microvesicle capture filter device. The capture filter device can accommodate volumes up to 13 mL so an additional spin was necessary for some samples to filter the entire volume. In other embodiments, the capture filter device can accommodate about 5 mL to about 10 mL, about 10 mL to about 15 mL, about 15 mL to about 20 mL, or any volume in between. After one or two 15 minute spins at 800×g, the tip of the filter device was removed and placed on a holder above a 96-well plate. In other embodiments, the spin or spins are performed for about 5 minutes to about 10 minutes, about 10 minutes to about 15 minutes, about 15 minutes to about 20 minutes, or any time in between. In other embodiments, the spin is at about 200×g to about 400×g, about 400×g to about 600×g, about 600×g to about 800×g, about 800×g to about 1,000×g, or any speed in between. The tip encases the filter which has the exosome and microvesicles captured on the membranes. lx lysis buffer is applied to the filter and incubated for 10 minutes at 37° C. In other embodiments, incubation may be from about 5 minutes to about 30 minutes, about 5 minutes to about 25 minutes, about 5 minutes to about 20 minutes, about 5 minutes to about 15 minutes, about 5 minutes to 10 about minutes, or any time in between. In other embodiments, incubation may be performed at about 30° C. to about 45° C., about 30° C. to about 44° C., about 30° C. to about 43° C., about 30° C. to about 42° C., about 30° C. to about 41° C., about 30° C. to about 40° C., about 30° C. to about 39° C., about 30° C. to about 38° C., about 30° C. to about 37° C., or any time in between. Following the incubation, the tip and holder is placed above an oligo(dT)-coated plate and a 5 minute spin at 2000×g transfers the lysate from the filter tip to the well. In other embodiments, the spin or spins are performed for about 1 minute to about 5 minutes, about 5 minutes to about 10 minutes, about 10 minutes to about 15 minutes, about 15 minutes to about 20 minutes, or any time in between. In other embodiments, the spin is at about 100×g to about 200×g, about 200×g to about 400×g, about 400×g to about 600×g, about 600×g to about 800×g, about 800×g to about 1,000×g, or any speed in between. Samples were hybridized overnight at about 4° C. To confirm that most exosomes and microvesicles were removed from the tampon in the step before, 1 mL 1× lysis buffer was added to the expunged tampon. The tampon was incubated at 37° C. for 10 minutes and expunged using the syringe method. In other embodiments, incubation may be from about 5 minutes to about 30 minutes, about 5 minutes to about 25 minutes, about 5 minutes to about 20 minutes, about 5 minutes to about 15 minutes, about 5 minutes to about 10 minutes, or any time in between. In other embodiments, incubation may be performed at about 30° C. to about 45° C., about 30° C. to about 44° C., about 30° C. to about 43° C., about 30° C. to about 42° C., about 30° C. to about 41° C., about 30° C. to about 40° C., about 30° C. to about 39° C., about 30° C. to about 38° C., about 30° C. to about 37° C., or any temperature in between. This lysate was then applied to the oligo(dT)-coated plate and hybridized in a similar manner as above.

Following mRNA hybridization at 4° C. overnight, the wells of the oligo(dT)-coated plate were washed with Wash Buffer A and B three times each. After removal of all traces of Wash Buffer B, 30 uL cDNA master mix with random primers was added to each well and incubated at 37° C. for 1 hour. Two μL of cDNA reactions were added to Sso Advance Universal (Bio-Rad) reaction mix with ACTB primers. The qPCR reaction was performed under standard conditions using ABI ViiA7 instrument and software.

### Example 2. Scanning Electron Microscopy of Exosomes and Microvesicles in Cervical-Vaginal Fluids Collected by Tampon

To confirm the presence of exosomes and microvesicles in cervical-vaginal fluids collected by tampon, scanning electron microscopy of the fluids were analyzed using phosphotungstic acid staining and immunoelectron microscopy using CD9 and CD63 antibodies.

In this example, a tampon worn for 7 hours overnight during a non-menstrual period by a healthy volunteer was a source of cervical-vaginal fluid. In other embodiments, the tampon can be worn for about 30 minutes to about 1 hour, about 1 hour to about 2 hours, about 2 hours to about 3 hours, about 3 hours to about 4 hours, about 4 hours to about 5 hours, about 6 hours to about 7 hours, about 7 hours to about 8 hours, about 8 hours to about 9 hours, or any time in between. The tampon was removed and placed at 4° C.-8° C. for 4 hours before the addition of 20 mL PBS. In other embodiments, the tampon can be placed at 4° C.-8° C. for about 1 hour to about 2 hours, about 2 hours to about 3 hours, about 3 hours to about 4 hours, or any time in between. In other embodiments PBS can be added in about 5 mL to about 10 mL, about 10 mL to about 15 mL, about 15 mL to about 20 mL, about 20 mL to about 25 mL, about 25 mL to about 30 mL, or any time in between. Following incubation for 5 minutes to allow for complete release of the cells, exosomes and microvesicles from the fibers, fluid was expunged from the tampon using a 20 cc syringe. In other embodiments, the fluid can be expunged using a about 5 cc to about 10 cc syringe, about 10 cc to about 20 cc syringe, about 20 cc to about 50 cc syringe, or about 50 cc to about 100 cc syringe. In other embodiments, incubation can be for about 5 minutes to about 10 minutes, about 10 minutes to about 15 minutes, about 15 minutes to about 20 minutes, about 20 minutes to about 25 minutes, about 25 minutes to about 30 minutes, or any time in between. Half of the volume (approximately 10 mL), was used in this example. A low speed spin of 2000×g for 15 minutes was used to remove cells and debris. In other embodiments, the spin can be performed for about 5 minutes to about 10 minutes, about 10 minutes to about 15 minutes, about 15 minutes to about 20 minutes, or any time in between. In other embodiments, the spin is at about 200×g to about 400×g, about 400×g to about 600×g, about 600×g to about 800×g, about 800×g to about 1,000×g, or any speed in between. A secondary spin at 10,000×g for 30 minutes was used to remove apoptotic bodies. In other embodiments, the second spin can be performed for about 10 minutes to about 15 minutes, about 15 minutes to about 20 minutes, about 20 minutes to about 25 minutes, about 25 minutes to about 30 minutes, about 30 minutes to about 35 minutes, about 35 minutes to about 40 minutes, or any time in between. In other embodiments, the spin is at about 1,000×g to about 5,000×g, about 5,000×g to about 6,000×g, about 6,000×g to about 7,000×g, about 7,000×g to about 8,000×g, about 8,000×g to about 9,000×g, about 9,000×g to about 10,000×g, about 10,000×g to about 11,000×g, about 11,000×g to about 12,000×g, about 12,000×g to about 13,000×g, about 13,000×g to about 14,000×g, about 14,000×g to about 15,000×g, about 15,000×g to about 20,000×g, or any speed in between. The supernatant was then transferred to an ultracentrifuge tube and spun at 110,000×g for 1 hour. The supernatant was then removed and PBS was added to wash the pellet. A second ultracentrifugation spin was done at 110,000×g for 1 hour. In other embodiments, the spin is at about 10,000×g to about 50,000×g, about 50,000×g to about 60,000×g, about 60,000×g to about 70,000×g, about 70,000×g to about 80,000×g, about 80,000×g to about 90,000×g, about 90,000×g to about 100,000×g, about 100,000×g to about 110,000×g, about 110,000×g to about 120,000×g, about 120,000×g to about 130,000×g, about 130,000×g to about 140,000×g, about 140,000×g to about 150,000×g, about 150,000×g to about 200,000×g, or any speed in between. The PBS was then removed and the pellet was resuspended in 100 μL PBS with heating at 37° C. for 15 minutes. In other embodiments, incubation may be from about 5 minutes to about 30 minutes, about 5 minutes to about 25 minutes, about 5 minutes to about 20 minutes, about 5 minutes to about 15 minutes, about 5 minutes to about 10 minutes, or any time in between. In other embodiments, incubation may be performed at about 30° C. to about 45° C., about 30° C. to about 44° C., about 30° C. to about 43° C., about 30° C. to about 42° C., about 30° C. to about 41° C., about 30° C. to about 40° C., about 30° C. to about 39° C., about 30° C. to about 38° C., about 30° C. to about 37° C., or any temperature in between.

Thirty μL of the resuspended sample was applied to a copper grid 200 mesh for 15 minutes. In other embodiments, the copper grid is about 50 mesh to about 100 mesh, about 100 mesh to about 150 mesh, about 150 mesh to about 200 mesh, about 200 mesh to about 250 mesh, about 250 mesh to about 300 mesh. In other embodiments, the sample was applied for about 5 minutes to about 10 minutes, about 10 minutes to about 15 minutes, about 15 minutes to about 20 minutes, about 20 minutes to about 25 minutes, about 25 minutes to about 30 minutes, or any time in between. In other embodiments, the tampon was placed for about 1 hour to about 2 hours, about 2 hours to about 3 hours, about 3 hours to about 4 hours, about 4 hours to about 5 hours, about 5 hours to about 6 hours, about 6 hours to about 7 hours, about 7 hours to about 8 hours, or any time in between. The grid was washed three times in PBS using the drop method. Sample was then fixed in 4% paraformaldehyde for 10 minutes. Grid was washed 3 times with PBS before incubation with 0.05M glycine/PBS for 15 minutes. Samples were blocked with 0.5% BSA for 15 minutes before incubation in 1:100 dilution CD9 or CD63 monoclonal antibody for 60 minutes. Grids were then washed 6 times in PBS before incubation in 1:100 dilution of gold conjugated goat anti-rabbit antibody for 60 minutes. Following incubation, grids were washed 6 times in PBS before successive dehydration steps from 40% to 98% ethyl alcohol. Grids were air dried overnight before scanning electron microscopy.

For staining with phosphotungstic acid, 30 μL of the resuspended cervical-vaginal fluid sample was applied to a copper grid 200 mesh for 15 minutes. The grid was washed three times in PBS using the drop method. Sample was then stained in 2% phosphotungstic acid for 5 seconds and washed immediately with water 3 times for 5 seconds each. Grids were air dried overnight before scanning electron microscopy.

### Example 3. RNA Sequencing of Cervical-Vaginal Fluid Exosomes and Microvesicles Demonstrate mRNA Expression Profiles Involved in Keratinocyte Differentiation, Epidermal and Epithelial Cell Differentiation

In this example, a tampon worn for 7 hours overnight during a non-menstrual period by a healthy volunteer was a source of cervical-vaginal fluid. In other embodiments, the tampon can be worn for between about 30 minutes to about 1 hour, about 1 hour to about 2 hours, about 2 hours to about 3 hours, about 3 hours to about 4 hours, about 4 hours to about 5 hours, about 6 hours to about 7 hours, about 7 hours to about 8 hours, about 8 hours to about 9 hours, or any time in between. The tampon was removed and placed at <8° C. for 4 hours before the addition of 20 mL PBS. In other embodiments, the tampon can be placed at about 0° C. to about 1° C., about 1° C. to about 2° C., about 2° C. to about 3° C., about 3° C. to about 4° C., about 4° C. to about 5° C., about 5° C. to about 6° C., about 6° C. to about 7° C., about 7° C. to about 8° C., or any time in between. Following incubation for 5 minutes to allow for complete release of the cells, exosomes and microvesicles from the fibers, fluid was expunged from the tampon using a 20 cc syringe. In other embodiments, the fluid can be expunged using an about 5 cc to about 10 cc syringe, about 10 cc to about 20 cc syringe, about 20 cc to about 50 cc syringe, or about 50 cc to about 100 cc syringe. In other embodiments, incubation can be for about 5 minutes to about 10 minutes, about 10 minutes to about 15 minutes, about 15 minutes to about 20 minutes, about 20 minutes to about 25 minutes, about 25 minutes to about 30 minutes, or any time in between. Half of the volume (approximately 10 mL), was used in this example. A low speed spin of 2000×g for 15 minutes was used to removed cells and debris. In other embodiments, the spin is performed for about 5 minutes to about 10 minutes, about 10 minutes to about 15 minutes, about 15 minutes to about 20 minutes, or any time in between. In other embodiments, the spin is at about 200×g to about 400×g, about 400×g to about 600×g, about 600×g to about 800×g, about 800×g to about 1,000×g, or any speed in between. This excess fluid was applied to an exosome and microvesicle capture filter device. After a 15 minute spin at 800×g, the tip of the filter device was removed and placed on a holder above a 96-well plate. In other embodiments, the spin is performed for about 5 minutes to about 10 minutes, about 10 minutes to about 15 minutes, about 15 minutes to about 20 minutes, or any time in between. In other embodiments, the spin is at about 200×g to about 400×g, about 400×g to about 600×g, about 600×g to about 800×g, about 800×g to about 1,000×g, or any speed in between. The tip encases the filter which has the exosome and microvesicles captured on the membranes. 1× lysis buffer is applied to the filter and incubated for 10 minutes at 37° C. Following the incubation, the tip and holder is placed above an oligo(dT)-coated plate and a 5 minute spin at 2000×g transfers the lysate from the filter tip to the well of the plate. In some embodiments, the spin is performed at about 1,000×g to about 1,500×g, about 1,500×g to about 2,000×g, about 2,000×g to about 2,500×g, about 2,500×g to about 3,000×g, or any speed in between. In some embodiments, the spin is performed for about 1 minute to about 5 minutes, about 5 minutes to about 10 minutes, about 10 minutes to about 15 minutes, about 15 minutes to about 20 minutes, or any time in between. Samples were hybridized overnight at 4° C. Following mRNA hybridization at 4° C. overnight, the wells of the oligo(dT)-coated plate were washed with Wash Buffer A and B 3 times each. The mRNA captured on the plate was then eluted using 60 Elution buffer and heating at 65° C. for 5 minutes.

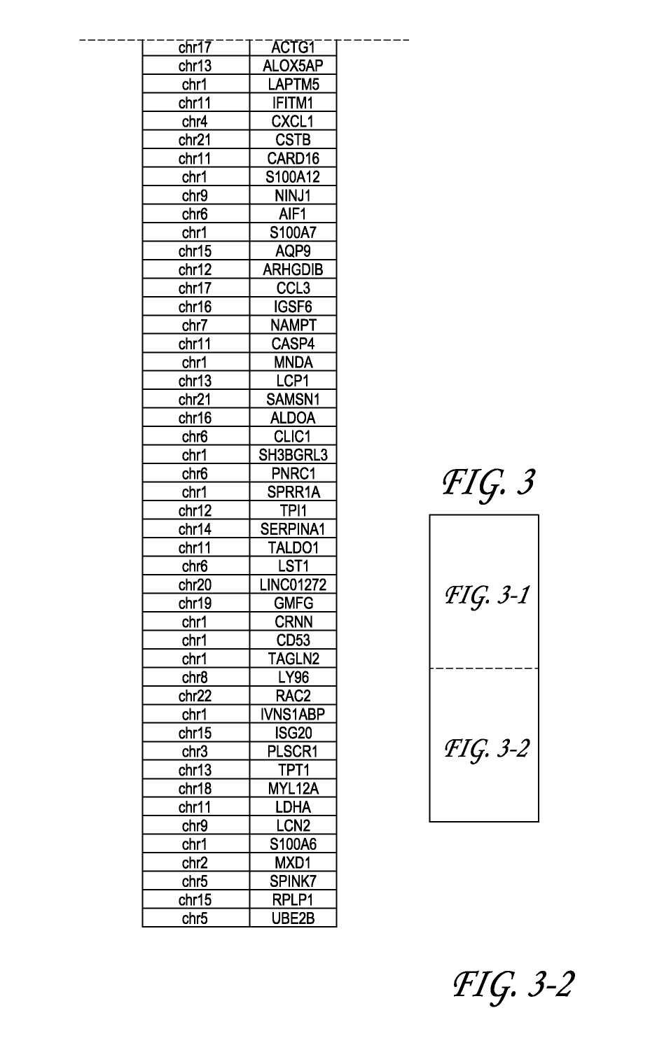

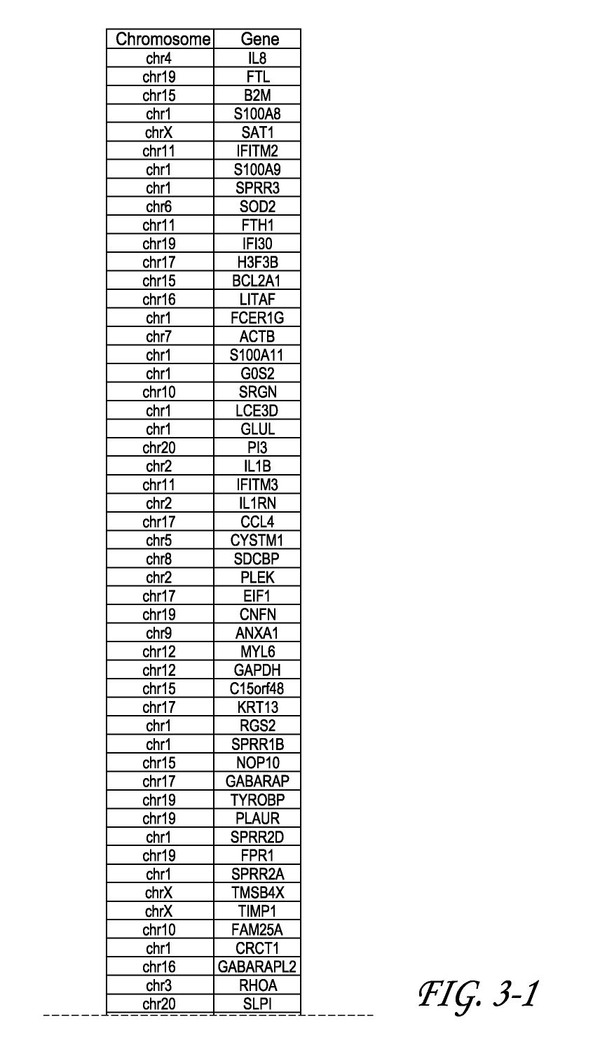

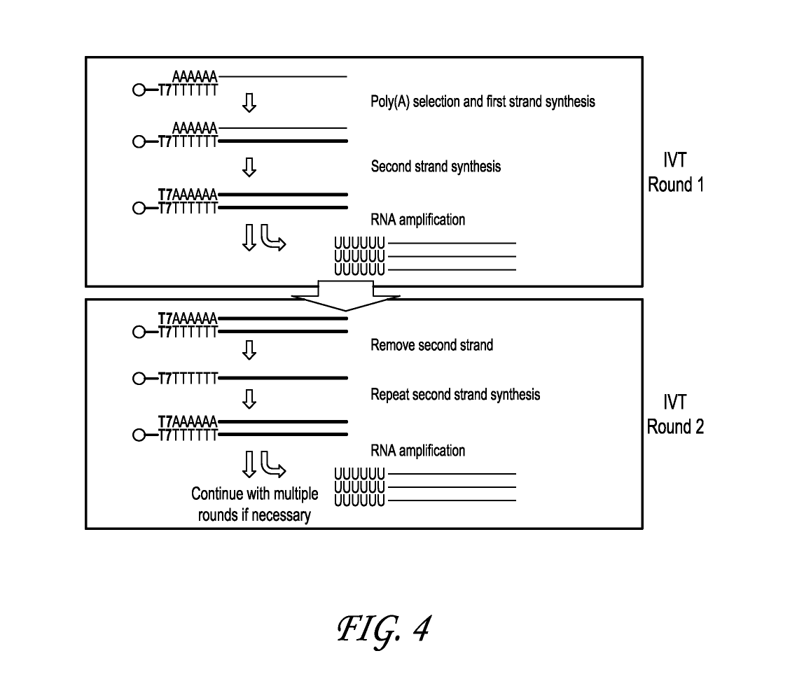

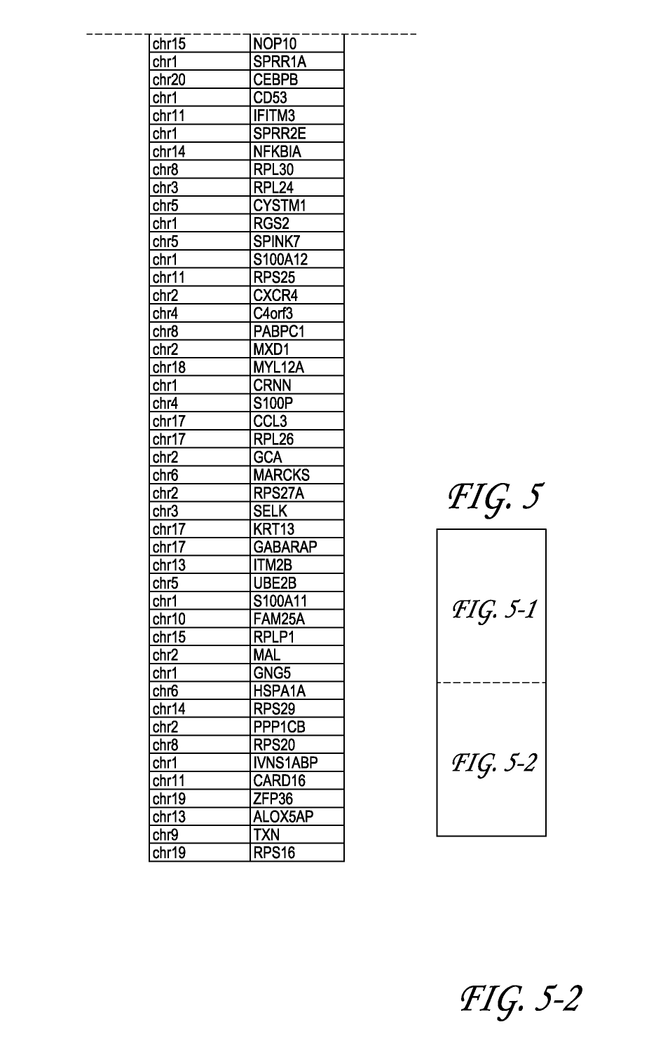

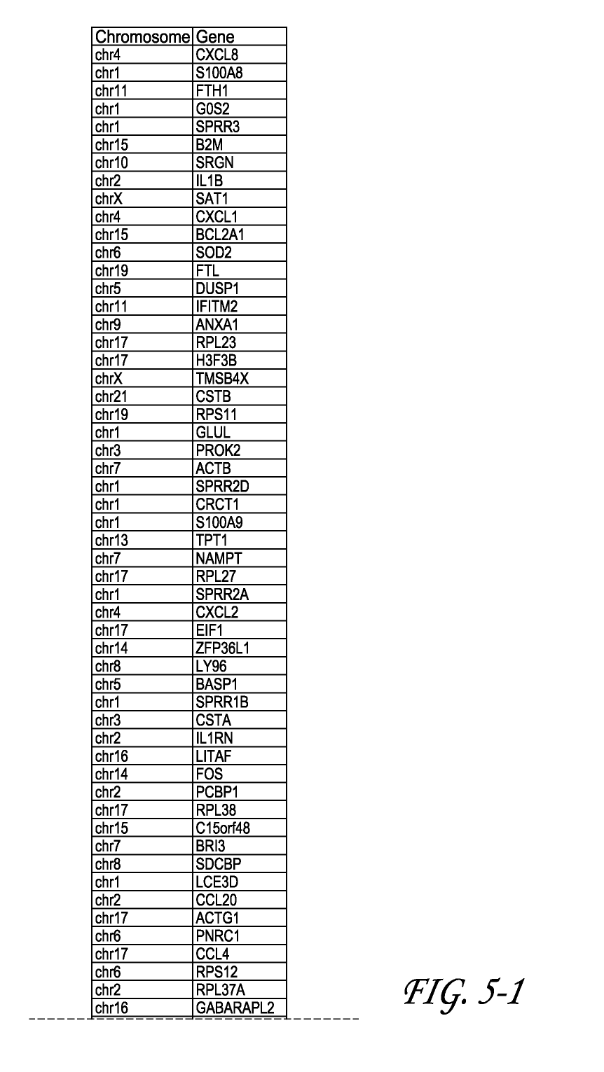

This eluted mRNA was used as the starting input for an RNA sequencing library preparation method from a commercial vendor. The Clontech Single Cell kit and Low Input Library Preparation kit were used to prepare double stranded cDNA library for RNA sequencing on the Illumina HiSeq 2500 instrument. A single read 50 run was performed. Highly expressed mRNAs are listed in FIG. 3. The main functional pathway clusters included keratinocyte differentiation, epidermal cell differentiation, epithelial cell differentiation, epithelium development, repeat:3, repeat:1, repeat:2, antigen processing and presentation of exogenous peptide antigen, and calcium-binding proteins.

An alternative method to prepare RNA sequencing libraries was also examined. Due to the low quantity of sample, an in-plate T7 RNA amplification was performed on the mRNA captured from the cervical-vaginal fluid exosome and microvesicle-containing fluid prior to library preparation. In this example, cervical-vaginal fluid was obtained as described above. A low speed spin of 2000×g for 15 minutes was used to remove cells and debris. This excess fluid was applied to an exosome and microvesicle capture filter device. After a 15 minute spin at 800×g, the tip of the filter device was removed and placed on a holder above a 96-well plate. The tip encases the filter which has the exosome and microvesicles captured on the membranes. 1× lysis buffer is applied to the filter and incubated for 10 minutes at 37° C. Following the incubation, the tip and holder is placed above a T7 oligo containing (dT)-coated plate (5′NH2-AGCTGAATTCGCGGCCGCTAATACGACTCACTATAGGGAGA(dT)18-3′) and a 5 minute spin at 2000×g transfers the lysate from the filter tip to the well of the plate. Samples were hybridized overnight at 4° C. Following mRNA hybridization at 4° C. overnight, the wells of the T7 oligo(dT)-coated plate were washed with Wash Buffer A and B 3 times each. A first and second strand cDNA synthesis was performed to create double-stranded DNA containing the T7 promoter sequence upstream of the mRNA sequence. A first round in vitro transcription reaction was performed for 17 hours using the T7 Megascript kit (Life Technologies). The amplified RNA was removed and stored at −80° C. while a second round in vitro transcription reaction was performed for an additional 17 hours. For the additional in vitro transcription reaction, the existing second strand was removed and a new second strand cDNA was synthesized. The amplified RNA from both the first and second in vitro transcription reactions was combined and purified using RNeasy MinElute Cleanup Kit (Qiagen). This RNA was then used as the starting material for the TruSeq RNA sample preparation v2 (Illumina). Double stranded cDNA was prepared, end repaired, adenylated and then ligated with Illumina barcoded adapters. The products were purified and PCR enriched using up to 15 cycles. Data was generated using single read (SR) 50 cycles plus index read sequencing on a Illumina HiSeq 2500 with v3 chemistry.

It is contemplated that various combinations or subcombinations of the specific features and aspects of the embodiments disclosed above may be made and still fall within one or more of the inventions. Further, the disclosure herein of any particular feature, aspect, method, property, characteristic, quality, attribute, element, or the like in connection with an embodiment can be used in all other embodiments set forth herein. Accordingly, it should be understood that various features and aspects of the disclosed embodiments can be combined with or substituted for one another in order to form varying modes of the disclosed inventions. Thus, it is intended that the scope of the present inventions herein disclosed should not be limited by the particular disclosed embodiments described above. Moreover, while the invention is susceptible to various modifications, and alternative forms, specific examples thereof have been shown in the drawings and are herein described in detail. It should be understood, however, that the invention is not to be limited to the particular forms or methods disclosed, but to the contrary, the invention is to cover all modifications, equivalents, and alternatives falling within the spirit and scope of the various embodiments described and the appended claims. Any methods disclosed herein need not be performed in the order recited. The methods disclosed herein include certain actions taken by a practitioner; however, they can also include any third-party instruction of those actions, either expressly or by implication. For example, actions such as “treating a subject for a gynecological disease or condition” include “instructing the administration of treatment of a subject for a gynecological disease or condition.”

Conditional language, such as, among others, “can,” “could,” “might,” or “may,” unless specifically stated otherwise, or otherwise understood within the context as used, is generally intended to convey that certain embodiments include, while other embodiments do not include, certain features, elements and/or steps. Thus, such conditional language is not generally intended to imply that features, elements and/or steps are in any way required for one or more embodiments.

Terms, such as, “first”, “second”, “third”, “fourth”, “fifth”, “sixth”, “seventh”, “eighth”, “ninth”, “tenth”, or “eleventh” and more, unless specifically stated otherwise, or otherwise understood within the context as used, are generally intended to refer to any order, and not necessarily to an order based on the plain meaning of the corresponding ordinal number. Therefore, terms using ordinal numbers may merely indicate separate individuals and may not necessarily mean the order therebetween. Accordingly, for example, first and second biomarkers used in this application may mean that there are merely two sets of biomarkers. In other words, there may not necessarily be any intention of order between the “first” and “second” sets of data in any aspects.

The ranges disclosed herein also encompass any and all overlap, sub-ranges, and combinations thereof. Language such as “up to,” “at least,” “greater than,” “less than,” “between,” and the like includes the number recited. Numbers preceded by a term such as “about” or “approximately” include the recited numbers. For example, “about 10 nanometers” includes “10 nanometers.”

What is claimed is:

1. A method of collecting cervical-vaginal fluids and identifying biomarkers for gynecological diseases and conditions, comprising:
(I) obtaining cervical-vaginal fluids by tampon usage;
(II) isolating one or more of membrane particles, cells, exosomes, exosome-like vesicles, and microvesicles from said tampon containing cervical-vaginal fluids; and
(III) detecting expression of a biomarker composed of one or more of the following:
IL8, FTL, B2M, S100A8, SAT1, IFITM2, S100A9, SPRR3, SOD2, FTH1, IFI30, H3F3B, BCL2A1, LITAF, FCER1G, ACTB, S100A1, GOS2, SRGN, LCE3D, GLUL, PI3, IL1B, IFITM3, IL1RN, CCL4, CYSTM1, SDCBP, PLEK, EIF1, CNFN, ANXA1, MYL6, GAPDH, C15orf48, KRT13, RGS2, SPRR1B, NOP10, GABARAP, TYROBP, PLAUR, SPRR2D, FPR1, SPRR2A, TMSB4X, TIMP1, FAM25A, CRCT1, GABARAPL2, RHOA, SLPI, ACTG1, ALOX5AP, LAPTM5, IFITM1, CXCL1, CSTB, CARD16, S100A12, NINJ1, AIF1, S100A7, AQP9, ARHGDIB, CCL3, IGSF6, NAMPT, CASP4, MNDA, LCP1, SAMSN1, ALDOA, CLIC1, SH3BGRL3, PNRC1, SPRR1A, TPI1, SERPINA1, TALDO1, LST1, LINC01272, GMFG, CRNN, CD53, TAGLN2, LY96, RAC2, IVNS1ABP, ISG20, PLSCR1, TPT1, MYL12A, LDHA, LCN2, S100A6, MXD1, SPINK7, RPLP1, UBE2B, CXCL8, DUSP1, RPL23, RPS11, PROK2, RPL27, CXCL2, ZFP36L1, BASP1, CSTA, FOX, PCBP1, RPL38, BRI3, SDCBP, CCL20, RPS12, RPL37A, CEBPB, SPRR2E, NFKBIA, RPL30, RPL24, CYSTM1, RGS2, RPS25, CXCR4, C4orf3, PABPC1, S100P, RPL26, GCA, MARCKS, RPS27A, SELK, ITM2B, MAL, HSPA1A, RPS29, PPP1CB, RPS20, IVNS1ABP, ZFP36, and TXN by a method comprising:
(a) liberating RNA from the isolated membrane particles, cells, exosomes, exosome-like vesicles, and/or microvesicles;
(b) contacting the liberated RNA with a reverse transcriptase to generate complementary DNA (cDNA); and
(c) contacting said cDNA with sense and antisense primers that are specific for the biomarker of the gynecological disease or condition and a DNA polymerase in order to generate amplified DNA.

2. The method of claim 1, wherein said isolating cervical-vaginal fluids comprises wearing a tampon for up to 8 hours during non-menstrual periods.

3. The method of claim 2, wherein said tampon is composed of a combination of cotton and rayon fibers that can absorb up to 6 g.

4. The method of claim 1, wherein said isolating cervical-vaginal fluids comprises adding excess buffer to the tampon to release one or more membrane particles, cells, exosomes, exosome-like vesicles, and/or microvesicles.

5. The method of claim 4 wherein excess buffer is between 10 to 30 mL.

6. The method of claim 4, wherein said excess buffer is composed of pH greater than 4 and less than 10 and not containing any detergent.

7. The method of claim 1, wherein said isolating cervical-vaginal fluids comprises using physical forces including a syringe or syringe-like device, centrifugation, shaking, air or liquid pressure to expunge fluids and/or membrane particles, cells, exosomes, exosome-like vesicles, and/or microvesicles from the tampon.

8. The method of claim 1, wherein filtration traps said one or more of membrane particles, exosomes, exosome-like vesicles, and microvesicles on a filter device.

9. The method of claim 1, wherein centrifugation isolates one or more of membrane particles, exosomes, exosome-like vesicles, and microvesicles.

10. A method of collecting cervical-vaginal fluids and identifying biomarkers for gynecological diseases and conditions, comprising:
(I) obtaining cervical-vaginal fluids by tampon usage;
(II) isolating one or more of membrane particles, cells, exosomes, exosome-like vesicles, and microvesicles from said tampon containing cervical-vaginal fluids; and
(III) identifying mRNA expression profile or mutations by a method comprising:
(a) liberating RNA from the isolated membrane particles, cells, exosomes, exosome-like vesicles, and/or microvesicles;
(b) preparing double-stranded cDNA library for RNA sequencing; and
(c) performing RNA sequencing.

11. The method of claim 10, wherein said isolating cervical-vaginal fluids comprises wearing a tampon for up to 8 hours during non-menstrual periods.

12. The method of claim 11, wherein said tampon is composed of a combination of cotton and rayon fibers that can absorb up to 6 g.

13. The method of claim 10, wherein said isolating cervical-vaginal fluids comprises adding excess buffer to the tampon to release one or more membrane particles, cells, exosomes, exosome-like vesicles, and/or microvesicles.

14. The method of claim 13 wherein excess buffer is between 10 to 30 mL.

15. The method of claim 13, wherein said excess buffer is composed of pH greater than 4 and less than 10 and not containing any detergent.

16. The method of claim 10, wherein said isolating cervical-vaginal fluids comprises using physical forces including a syringe or syringe-like device, centrifugation, shaking, air or liquid pressure to expunge fluids and/or membrane particles, cells, exosomes, exosome-like vesicles, and/or microvesicles from the tampon.

17. The method of claim 10, wherein filtration traps said one or more of membrane particles, exosomes, exosome-like vesicles, and microvesicles on a filter device.

18. The method of claim 10, wherein centrifugation isolates one or more of membrane particles, exosomes, exosome-like vesicles, and microvesicles.

19. A method of collecting vaginal fluids and analyzing biomarkers to determine whether a subject is suffering from a gynecological disease or condition, the method comprising:
(I) obtaining a tampon comprising cervical-vaginal fluids that has been used by a subject;
(II) isolating one or more of membrane particles, cells, exosomes, exosome-like vesicles, and microvesicles from said tampon;
(III) detecting expression of a biomarker associated with a gynecological disease or condition, wherein said biomarker is selected from the group consisting of: IL8, FTL, B2M, S100A8, SAT1, IFITM2, S100A9, SPRR3, SOD2, FTH1, IFI30, H3F3B, BCL2A1, LITAF, FCER1G, ACTB, S100A1, GOS2, SRGN, LCE3D, GLUL, PI3, IL1B, IFITM3, IL1RN, CCL4, CYSTM1, SDCBP, PLEK, EIF1, CNFN, ANXA1, MYL6, GAPDH, C15orf48, KRT13, RGS2, SPRR1B, NOP10, GABARAP, TYROBP, PLAUR, SPRR2D, FPR1, SPRR2A, TMSB4X, TIMP1, FAM25A, CRCT1, GABARAPL2, RHOA, SLPI, ACTG1, ALOX5AP, LAPTM5, IFITM1, CXCL1, CSTB, CARD16, S100A12, NINJ1, AIF1, S100A7, AQP9, ARHGDIB, CCL3, IGSF6, NAMPT, CASP4, MNDA, LCP1, SAMSN1, ALDOA, CLIC1, SH3BGRL3, PNRC1, SPRR1A, TPI1, SERPINA1, TALDO1, LST1, LINC01272, GMFG, CRNN, CD53, TAGLN2, LY96, RAC2, IVNS1ABP, ISG20, PLSCR1, TPT1, MYL12A, LDHA, LCN2, S100A6, MXD1, SPINK7, RPLP1, UBE2B, CXCL8, DUSP1, RPL23, RPS11, PROK2, RPL27, CXCL2, ZFP36L1, BASP1, CSTA, FOX, PCBP1, RPL38, BRI3, SDCBP, CCL20, RPS12, RPL37A, CEBPB, SPRR2E, NFKBIA, RPL30, RPL24, CYSTM1, RGS2, RPS25, CXCR4, C4orf3, PABPC1, S100P, RPL26, GCA, MARCKS, RPS27A, SELK, ITM2B, MAL, HSPA1A, RPS29, PPP1CB, RPS20, IVNS1ABP, ZFP36, and TXN by a method comprising:
(a) liberating RNA from said isolated membrane particles, cells, exosomes, exosome-like vesicles, and/or microvesicles;
(b) contacting said liberated RNA with a reverse transcriptase to generate complementary DNA (cDNA); and
(c) contacting said cDNA with sense and antisense primers that are specific for said biomarker and a DNA polymerase in order to generate amplified DNA;
(IV) comparing said expression of said biomarker to expression of said biomarker in a control sample; and
(V) determining that said subject is suffering from a gynecological disease or condition when expression of said biomarker is greater when compared to said expression in said control sample.

20. The method of claim 1, wherein said tampon was used by said subject for up to 8 hours during a non-menstrual time-period prior to said obtaining step.

21. The method of claim 1, wherein said tampon is composed of a combination of cotton and rayon fibers that can absorb up to 6 g.

22. The method of claim 1, wherein said isolation of said membrane particles, cells, exosomes, exosome-like vesicles, and/or microvesicles from said tampon containing cervical-vaginal fluids comprises adding an excess of a buffer to the tampon to release said membrane particles, cells, exosomes, exosome-like vesicles, and/or microvesicles.

23. The method of claim 4, wherein between 10 to 30 mL of said excess buffer is added.

24. The method of claim 4, wherein said excess buffer is composed of pH greater than 4 and less than 10, and wherein said excess buffer does not comprise a detergent.

25. The method of claim 1, wherein said isolating of said membrane particles, cells, exosomes, exosome-like vesicles, and/or microvesicles from said tampon comprising cervical-vaginal fluids comprises using one or more types of physical force selected from the group consisting of using a syringe or syringe-like device, centrifugation, shaking, air pressure, and liquid pressure.

26. The method of claim 1, wherein said isolating of said membrane particles, cells, exosomes, exosome-like vesicles, and/or microvesicles from said tampon comprising cervical-vaginal fluids comprises using a filter device.

27. The method of claim 1, wherein said isolating of said membrane particles, cells, exosomes, exosome-like vesicles, and/or microvesicles from said tampon containing cervical-vaginal fluids comprises using centrifugation.

28. The method of claim 1, wherein said biomarker is selected from the group consisting of IL-8, FTL, B2M, S100A8, S100A9, SAT1, IFITM2, SPRR3, SOD2, FTH1, CXCL8, GOS2, SRGN, IL-1B, and CXCL1.

29. A method of collecting cervical-vaginal fluids from a subject to determine whether a subject has a gynecological disease or condition, the method comprising:
(I) obtaining a tampon comprising cervical-vaginal fluids that has been used by a subject;
(II) isolating one or more of membrane particles, cells, exosomes, exosome-like vesicles, and microvesicles from said tampon;
(III) identifying an mRNA expression profile, wherein said mRNA expression profile is generated for one or more mRNA associated with a gynecological disease or condition, by a method comprising:
(a) liberating RNA from said isolated membrane particles, cells, exosomes, exosome-like vesicles, and/or microvesicles,
(b) preparing a double-stranded cDNA library for RNA sequencing from said RNA,
(c) performing sequencing of said cDNA,
(d) identifying said mRNA expression profile based on the results of said cDNA sequencing; and
(IV) determining whether a subject has a gynecological disease or condition based on comparing said mRNA expression profile of said subject to a control mRNA expression profile, wherein said expression of said biomarker is higher in said subject as compared to said control mRNA expression profile when said subject is suffering from a gynecological disease or condition.

30. The method of claim 29, wherein said tampon is used by said subject for up to 8 hours during a non-menstrual time-period.

31. The method of claim 29, wherein said tampon is composed of a combination of cotton and rayon fibers that can absorb up to 6 g.

32. The method of claim 29, wherein said isolation of said membrane particles, cells, exosomes, exosome-like vesicles, and/or microvesicles from said tampon containing cervical-vaginal fluids comprises adding an excess of a buffer to the tampon to release said membrane particles, cells, exosomes, exosome-like vesicles, and/or microvesicles.

33. The method of claim 32, wherein between 10 to 30 mL of said excess buffer is added.

34. The method of claim 32, wherein said excess buffer is composed of pH greater than 4 and less than 10, and wherein said excess buffer does not comprise a detergent.

35. The method of claim 29, wherein said isolating of said membrane particles, cells, exosomes, exosome-like vesicles, and/or microvesicles from said tampon containing cervical-vaginal fluids comprises using one or more types of physical force selected from the group of using a syringe or syringe-like device, centrifugation, shaking, air pressure, or liquid pressure.

36. The method of claim 29, wherein said isolating of said membrane particles, cells, exosomes, exosome-like vesicles, and/or microvesicles from said tampon containing cervical-vaginal fluids comprises using a filter device.

37. The method of claim 29, wherein said isolating of said membrane particles, cells, exosomes, exosome-like vesicles, and/or microvesicles from said tampon containing cervical-vaginal fluids comprises using centrifugation.

38. The method of claim 29, wherein said biomarker is selected from the group consisting of IL-8, FTL, B2M, S100A8, S100A9, SAT1, IFITM2, SPRR3, SOD2, FTH1, CXCL8, GOS2, SRGN, IL-1B, and CXCL1.

39. A method of treating a subject suffering from a gynecological disease or condition, comprising:
(I) ordering that a tampon with cervical-vaginal fluid that has been used by a subject is collected;
(II) ordering a test of said cervical-vaginal fluid of said tampon by a method comprising:
isolating one or more of membrane particles, cells, exosomes, exosome-like vesicles, and microvesicles from said cervical-vaginal fluid of said tampon;
liberating RNA from said isolated membrane particles, cells, exosomes, exosome-like vesicles, and/or microvesicles;
contacting said liberated RNA with a reverse transcriptase to generate complementary DNA (cDNA); and
contacting said cDNA with sense and antisense primers that are specific for one or more markers of gynecological disease or condition and a DNA polymerase in order to generate amplified DNA, wherein said one or more markers of gynecological disease or condition is chosen from:
IL8, FTL, B2M, S100A8, SAT1, IFITM2, S100A9, SPRR3, SOD2, FTH1, IFI30, H3F3B, BCL2A1, LITAF, FCER1G, ACTB, S100A1, GOS2, SRGN, LCE3D, GLUL, PI3, IL1B, IFITM3, IL1RN, CCL4, CYSTM1, SDCBP, PLEK, EIF1, CNFN, ANXA1, MYL6, GAPDH, C15orf48, KRT13, RGS2, SPRR1B, NOP10, GABARAP, TYROBP, PLAUR, SPRR2D, FPR1, SPRR2A, TMSB4X, TIMP1, FAM25A, CRCT1, GABARAPL2, RHOA, SLPI, ACTG1, ALOX5AP, LAPTM5, IFITM1, CXCL1, CSTB, CARD16, S100A12, NINJ1, AIF1, S100A7, AQP9, ARHGDIB, CCL3, IGSF6, NAMPT, CASP4, MNDA, LCP1, SAMSN1, ALDOA, CLIC1, SH3BGRL3, PNRC1, SPRR1A, TPI1, SERPINA1, TALDO1, LST1, LINC01272, GMFG, CRNN, CD53, TAGLN2, LY96, RAC2, IVNS1ABP, ISG20, PLSCR1, TPT1, MYL12A, LDHA, LCN2, S100A6, MXD1, SPINK7, RPLP1, UBE2B, CXCL8, DUSP1, RPL23, RP S11, PROK2, RPL27, CXCL2, ZFP36L1, BASP1, CSTA, FOX, PCBP1, RPL38, BRI3, SDCBP, CCL20, RPS12, RPL37A, CEBPB, SPRR2E, NFKBIA, RPL30, RPL24, CYSTM1, RGS2, RPS25, CXCR4, C4orf3, PABPC1, S100P, RPL26, GCA, MARCKS, RPS27A, SELK, ITM2B, MAL, HSPA1A, RPS29, PPP1CB, RPS20, IVNS1ABP, ZFP36, and TXN;
detecting the amount of expression of said one or more markers of gynecological disease or condition;
(III) obtaining the results of the test, wherein the test results indicate that said subject is suffering from a gynecological disease or condition when said expression of said marker is different than a control mRNA expression profile; and
(IV) treating the subject when said test results indicate that the subject is suffering from a gynecological disease or condition.

40. A method of directing treatment of a subject suffering from a gynecological condition or disease, comprising:
(I) receiving cervical-vaginal fluid collected from a tampon that has been used by a subject;
(II) detecting expression of at least one marker of gynecological disease or condition from the group consisting of:
IL8, FTL, B2M, S100A8, SAT1, IFITM2, S100A9, SPRR3, SOD2, FTH1, IFI30, H3F3B, BCL2A1, LITAF, FCER1G, ACTB, S100A1, GOS2, SRGN, LCE3D, GLUL, PI3, IL1B, IFITM3, IL1RN, CCL4, CYSTM1, SDCBP, PLEK, EIF1, CNFN, ANXA1, MYL6, GAPDH, C15orf48, KRT13, RGS2, SPRR1B, NOP10, GABARAP, TYROBP, PLAUR, SPRR2D, FPR1, SPRR2A, TMSB4X, TIMP1, FAM25A, CRCT1, GABARAPL2, RHOA, SLPI, ACTG1, ALOX5AP, LAPTM5, IFITM1, CXCL1, CSTB, CARD16, S100A12, NINJ1, AIF1, S100A7, AQP9, ARHGDIB, CCL3, IGSF6, NAMPT, CASP4, MNDA, LCP1, SAMSN1, ALDOA, CLIC1, SH3BGRL3, PNRC1, SPRR1A, TPI1, SERPINA1, TALDO1, LST1, LINC01272, GMFG, CRNN, CD53, TAGLN2, LY96, RAC2, IVNS1ABP, ISG20, PLSCR1, TPT1, MYL12A, LDHA, LCN2, S100A6, MXD1, SPINK7, RPLP1, UBE2B, CXCL8, DUSP1, RPL23, RPS11, PROK2, RPL27, CXCL2, ZFP36L1, BASP1, CSTA, FOX, PCBP1, RPL38, BRI3, SDCBP, CCL20, RPS12, RPL37A, CEBPB, SPRR2E, NFKBIA, RPL30, RPL24, CYSTM1, RGS2, RPS25, CXCR4, C4orf3, PABPC1, S100P, RPL26, GCA, MARCKS, RPS27A, SELK, ITM2B, MAL, HSPA1A, RPS29, PPP1CB, RPS20, IVNS1ABP, ZFP36, and TXN by a method comprising:
isolating one or more of membrane particles, cells, exosomes, exosome-like vesicles, and microvesicles from said cervical-vaginal fluid of said tampon;
liberating RNA from said isolated membrane particles, cells, exosomes, exosome-like vesicles, and/or microvesicles;
contacting said liberated RNA with a reverse transcriptase to generate complementary DNA (cDNA); and
contacting said cDNA with sense and antisense primers that are specific for said marker of gynecological disease in order to generate amplified DNA;
(III) identifying said subject as suffering from a gynecological disease or condition when said expression of said marker is different as compared to a control mRNA expression profile;
(IV) informing a physician that it would be appropriate to treat said subject if said expression indicates that said subject is suffering from a gynecological disease or condition.

41. A method of collecting vaginal fluids and analyzing biomarkers to determine whether a subject is suffering from a gynecological disease or condition, the method comprising:
(I) obtaining a tampon comprising cervical-vaginal fluids that has been used by a subject;
(II) isolating one or more of membrane particles, cells, exosomes, exosome-like vesicles, and microvesicles from said tampon;
(III) detecting expression of a biomarker associated with a gynecological disease or condition, wherein said biomarker is selected from the group consisting of: IL8, FTL, B2M, S100A8, SAT1, IFITM2, S100A9, SPRR3, SOD2, FTH1, IFI30, H3F3B, BCL2A1, LITAF, FCER1G, ACTB, S100A1, GOS2, SRGN, LCE3D, GLUL, PI3, IL1B, IFITM3, IL1RN, CCL4, CYSTM1, SDCBP, PLEK, EIF1, CNFN, ANXA1, MYL6, GAPDH, C15orf48, KRT13, RGS2, SPRR1B, NOP10, GABARAP, TYROBP, PLAUR, SPRR2D, FPR1, SPRR2A, TMSB4X, TIMP1, FAM25A, CRCT1, GABARAPL2, RHOA, SLPI, ACTG1, ALOX5AP, LAPTM5, IFITM1, CXCL1, CSTB, CARD16, S100A12, NINJ1, AIF1, S100A7, AQP9, ARHGDIB, CCL3, IGSF6, NAMPT, CASP4, MNDA, LCP1, SAMSN1, ALDOA, CLIC1, SH3BGRL3, PNRC1, SPRR1A, TPI1, SERPINA1, TALDO1, LST1, LINC01272, GMFG, CRNN, CD53, TAGLN2, LY96, RAC2, IVNS1ABP, ISG20, PLSCR1, TPT1, MYL12A, LDHA, LCN2, S100A6, MXD1, SPINK7, RPLP1, UBE2B, CXCL8, DUSP1, RPL23, RPS11, PROK2, RPL27, CXCL2, ZFP36L1, BASP1, CSTA, FOX, PCBP1, RPL38, BRI3, SDCBP, CCL20, RPS12, RPL37A, CEBPB, SPRR2E, NFKBIA, RPL30, RPL24, CYSTM1, RGS2, RPS25, CXCR4, C4orf3, PABPC1, S100P, RPL26, GCA, MARCKS, RPS27A, SELK, ITM2B, MAL, HSPA1A, RPS29, PPP1CB, RPS20, IVNS1ABP, ZFP36, and TXN by a method comprising:
(a) liberating RNA from said isolated membrane particles, cells, exosomes, exosome-like vesicles, and/or microvesicles;
(b) contacting said liberated RNA with a reverse transcriptase to generate complementary DNA (cDNA); and
(c) contacting said cDNA with sense and antisense primers that are specific for said biomarker and a DNA polymerase in order to generate amplified DNA; and
(IV) using a computer configured to receive data from step (III) and programmed to determine whether said expression of said biomarker indicates that said subject is suffering from a gynecological disease or condition.

42. A method of treating a subject suffering from a gynecological disease or condition, the method comprising:
(I) obtaining a tampon comprising cervical-vaginal fluids that has been used by a subject;
(II) isolating one or more of membrane particles, cells, exosomes, exosome-like vesicles, and microvesicles from said tampon;
(III) detecting expression of a biomarker associated with a gynecological disease or condition, wherein said biomarker is selected from the group consisting of: IL8, FTL, B2M, S100A8, SAT1, IFITM2, S100A9, SPRR3, SOD2, FTH1, IFI30, H3F3B, BCL2A1, LITAF, FCER1G, ACTB, S100A1, GOS2, SRGN, LCE3D, GLUL, PI3, IL1B, IFITM3, IL1RN, CCL4, CYSTM1, SDCBP, PLEK, EIF1, CNFN, ANXA1, MYL6, GAPDH, C15orf48, KRT13, RGS2, SPRR1B, NOP10, GABARAP, TYROBP, PLAUR, SPRR2D, FPR1, SPRR2A, TMSB4X, TIMP1, FAM25A, CRCT1, GABARAPL2, RHOA, SLPI, ACTG1, ALOX5AP, LAPTM5, IFITM1, CXCL1, CSTB, CARD16, S100A12, NINJ1, AIF1, S100A7, AQP9, ARHGDIB, CCL3, IGSF6, NAMPT, CASP4, MNDA, LCP1, SAMSN1, ALDOA, CLIC1, SH3BGRL3, PNRC1, SPRR1A, TPI1, SERPINA1, TALDO1, LST1, LINC01272, GMFG, CRNN, CD53, TAGLN2, LY96, RAC2, IVNS1ABP, ISG20, PLSCR1, TPT1, MYL12A, LDHA, LCN2, S100A6, MXD1, SPINK7, RPLP1, UBE2B, CXCL8, DUSP1, RPL23, RPS11, PROK2, RPL27, CXCL2, ZFP36L1, BASP1, CSTA, FOX, PCBP1, RPL38, BRI3, SDCBP, CCL20, RPS12, RPL37A, CEBPB, SPRR2E, NFKBIA, RPL30, RPL24, CYSTM1, RGS2, RPS25, CXCR4, C4orf3, PABPC1, S100P, RPL26, GCA, MARCKS, RPS27A, SELK, ITM2B, MAL, HSPA1A, RPS29, PPP1CB, RPS20, IVNS1ABP, ZFP36, and TXN by a method comprising:
(a) liberating RNA from said isolated membrane particles, cells, exosomes, exosome-like vesicles, and/or microvesicles;
(b) contacting said liberated RNA with a reverse transcriptase to generate complementary DNA (cDNA); and
(c) contacting said cDNA with sense and antisense primers that are specific for said biomarker and a DNA polymerase in order to generate amplified DNA;
(IV) comparing said expression of said biomarker to expression of said biomarker in a control sample;
(V) treating said subject for a gynecological disease or condition when expression of said biomarker is different when compared to said expression in said control sample.

43. A method of treating a subject suffering from a gynecological disease or condition, the method comprising:
(I) obtaining a tampon comprising cervical-vaginal fluids that has been used by a subject;
(II) isolating one or more of membrane particles, cells, exosomes, exosome-like vesicles, and microvesicles from said tampon;
(III) identifying an mRNA expression profile, wherein said mRNA expression profile is generated for one or more mRNA associated with a gynecological disease or condition, by a method comprising:
(a) liberating RNA from said isolated membrane particles, cells, exosomes, exosome-like vesicles, and/or microvesicles,
(b) preparing a double-stranded cDNA library for RNA sequencing from said RNA,
(c) performing sequencing of said cDNA,
(d) identifying said mRNA expression profile based on the results of said cDNA sequencing; and
(IV) treating said subject for a gynecological disease or condition when said mRNA expression profile of said subject is different when compared to a control mRNA expression profile.

44. The method of claim 43, wherein the difference in the expression profile of the subject as compared to a control mRNA expression profile is an increase in the expression of a biomarker associated with a gynecological disease or condition.

45. The method of claim 43, wherein the difference in the expression profile of the subject as compared to a control mRNA expression profile is a decrease in the expression of a biomarker associated with a gynecological disease or condition.

46. A method of treating a subject suffering from a gynecological disease or condition, the method comprising:
(I) obtaining a female hygiene product comprising cervical-vaginal fluids that has been used by a subject;
(II) isolating one or more of membrane particles, cells, exosomes, exosome-like vesicles, and microvesicles from said female hygiene product;
(III) detecting expression of a biomarker associated with a gynecological disease or condition, wherein said biomarker is selected from the group consisting of: IL8, FTL, B2M, S100A8, SAT1, IFITM2, S100A9, SPRR3, SOD2, FTH1, IFI30, H3F3B, BCL2A1, LITAF, FCER1G, ACTB, S100A1, GOS2, SRGN, LCE3D, GLUL, PI3, IL1B, IFITM3, IL1RN, CCL4, CYSTM1, SDCBP, PLEK, EIF1, CNFN, ANXA1, MYL6, GAPDH, C15orf48, KRT13, RGS2, SPRR1B, NOP10, GABARAP, TYROBP, PLAUR, SPRR2D, FPR1, SPRR2A, TMSB4X, TIMP1, FAM25A, CRCT1, GABARAPL2, RHOA, SLPI, ACTG1, ALOX5AP, LAPTM5, IFITM1, CXCL1, CSTB, CARD16, S100A12, NINJ1, AIF1, S100A7, AQP9, ARHGDIB, CCL3, IGSF6, NAMPT, CASP4, MNDA, LCP1, SAMSN1, ALDOA, CLIC1, SH3BGRL3, PNRC1, SPRR1A, TPI1, SERPINA1, TALDO1, LST1, LINC01272, GMFG, CRNN, CD53, TAGLN2, LY96, RAC2, IVNS1ABP, ISG20, PLSCR1, TPT1, MYL12A, LDHA, LCN2, S100A6, MXD1, SPINK7, RPLP1, UBE2B, CXCL8, DUSP1, RPL23, RPS11, PROK2, RPL27, CXCL2, ZFP36L1, BASP1, CSTA, FOX, PCBP1, RPL38, BRI3, SDCBP, CCL20, RPS12, RPL37A, CEBPB, SPRR2E, NFKBIA, RPL30, RPL24, CYSTM1, RGS2, RPS25, CXCR4, C4orf3, PABPC1, S100P, RPL26, GCA, MARCKS, RPS27A, SELK, ITM2B, MAL, HSPA1A, RPS29, PPP1CB, RPS20, IVNS1ABP, ZFP36, and TXN by a method comprising:
(a) liberating RNA from said isolated membrane particles, cells, exosomes, exosome-like vesicles, and/or microvesicles;
(b) contacting said liberated RNA with a reverse transcriptase to generate complementary DNA (cDNA); and
(c) contacting said cDNA with sense and antisense primers that are specific for said biomarker and a DNA polymerase in order to generate amplified DNA;
(IV) comparing said expression of said biomarker to expression of said biomarker in a control sample;
(V) treating said subject for a gynecological disease or condition when expression of said biomarker is different when compared to said expression in said control sample.

47. A method of collecting vaginal fluids and analyzing biomarkers to determine whether a subject is suffering from a gynecological disease or condition, the method comprising:
(A) having a female hygiene product comprising cervical-vaginal fluids that has been used by a subject placed into a fully automated machine for the machine to perform an assay comprising the following steps:
(1) isolating one or more of membrane particles, cells, exosomes, exosome-like vesicles, and microvesicles from said female hygiene product;
(2) detecting the mRNA expression profile of a biomarker associated with a gynecological disease or condition, wherein said biomarker is selected from the group consisting of: IL8, FTL, B2M, S100A8, SAT1, IFITM2, S100A9, SPRR3, SOD2, FTH1, IFI30, H3F3B, BCL2A1, LITAF, FCER1G, ACTB, S100A1, GOS2, SRGN, LCE3D, GLUL, PI3, IL1B, IFITM3, IL1RN, CCL4, CYSTM1, SDCBP, PLEK, EIF1, CNFN, ANXA1, MYL6, GAPDH, C15orf48, KRT13, RGS2, SPRR1B, NOP10, GABARAP, TYROBP, PLAUR, SPRR2D, FPR1, SPRR2A, TMSB4X, TIMP1, FAM25A, CRCT1, GABARAPL2, RHOA, SLPI, ACTG1, ALOX5AP, LAPTM5, IFITM1, CXCL1, CSTB, CARD16, S100A12, NINJ1, AIF1, S100A7, AQP9, ARHGDIB, CCL3, IGSF6, NAMPT, CASP4, MNDA, LCP1, SAMSN1, ALDOA, CLIC1, SH3BGRL3, PNRC1, SPRR1A, TPI1, SERPINA1, TALDO1, LST1, LINC01272, GMFG, CRNN, CD53, TAGLN2, LY96, RAC2, IVNS1ABP, ISG20, PLSCR1, TPT1, MYL12A, LDHA, LCN2, S100A6, MXD1, SPINK7, RPLP1, UBE2B, CXCL8, DUSP1, RPL23, RPS11, PROK2, RPL27, CXCL2, ZFP36L1, BASP1, CSTA, FOX, PCBP1, RPL38, BRI3, SDCBP, CCL20, RPS12, RPL37A, CEBPB, SPRR2E, NFKBIA, RPL30, RPL24, CYSTM1, RGS2, RPS25, CXCR4, C4orf3, PABPC1, S100P, RPL26, GCA, MARCKS, RPS27A, SELK, ITM2B, MAL, HSPA1A, RPS29, PPP1CB, RPS20, IVNS1ABP, ZFP36, and TXN by a method comprising:
(a) liberating RNA from said isolated membrane particles, cells, exosomes, exosome-like vesicles, and/or microvesicles;
(b) contacting said liberated RNA with a reverse transcriptase to generate complementary DNA (cDNA); and
(c) contacting said cDNA with sense and antisense primers that are specific for said biomarker and a DNA polymerase in order to generate amplified DNA; and
(B) determining whether a subject has a gynecological disease or condition based on comparing said mRNA expression profile of said subject to a control mRNA expression profile, wherein said expression of said biomarker is higher in said subject as compared to said control mRNA expression profile when said subject is suffering from a gynecological disease or condition.
